SEQUENCE LISTING (80 sequences)

SEQ ID NO: 1 - DNA, 1347 bp, Zea mays
atgtcgaggtgcttcccctacccgccaccggggtacgtgcggaacccagtggccgtggccgagccggagtcgaccgctaagctcctgaaagaaaaggaaaaagccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtctggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggagatcaggacctcttcctcaaaagaagagccctgcttctctggtaggaatgcagaagcagttcaagtgcaggatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 2 - DNA, 1347 bp, Zea mays; identical to SEQ ID NO: 1
  CDS (1)..(1347)

SEQ ID NO: 3 - protein, 448 aa, Zea mays
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQEIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 4 - DNA, 1347 bp, Zea mays
atgtcgaggtgcttcccctacccgccaccggggtacgtgcggaacccagtggccgtggccgagccggagtcgaccgctaagctcctgaaagaaaaggaaaaagccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtctggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggagatcaggacctctttctcaaaagaagagccctgcttctctggtaggaatgcagaagcagttcaagtgcaggatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 5 - DNA, 1347 bp, Zea mays; identical to SEQ ID NO: 4
  CDS (1)..(1347)

SEQ ID NO: 6 - protein, 448 aa, Zea mays
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQEIRTSFSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 7 - DNA, 1347 bp, Zea mays
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagatcccaagcagtgtgagacgtccaaacactcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgggactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcaggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgccttctatcaagcagcaggagatcaggacctcttcctcaaaagaagagccctgcttctctggtaggaatgcagaagctgttcaagtgcaggatactaagctctcccggtcagatatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 8 - DNA, 1347 bp, Zea mays; identical to SEQ ID NO: 7
  CDS (1)..(1347)

SEQ ID NO: 9 - protein, 448 aa, Zea mays
MSRCFPYPPPGYVRNPVAVAEPESTAKLLKEKEKAEKKKEKRSDRKDPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPSIKQQEIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 10 - DNA, 1347 bp, Zea mays
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagacccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcgatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 11 - DNA, 1347 bp, Zea mays; identical to SEQ ID NO: 10
  CDS (1)..(1347)

SEQ ID NO: 12 - protein, 448 aa, Zea mays
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVDGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQDIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 13 - DNA, 1347 bp, Zea mays
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccccgtcgggcaagcaaaattcaatccatcataagatgaatgtgagatctacctctggtcagcggagggtcgatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 14 - DNA, 1347 bp, Zea mays; identical to SEQ ID NO: 13
  CDS (1)..(1347)

SEQ ID NO: 15 - protein, 448 aa, Zea mays
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSPSGKQNSIHHKMNVRSTSGQRRVDGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQDIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 16 - DNA, 1347 bp, Zea mays
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagacccccaatcagctgttctggagaaaccaagggttcttgagcaaccattggtccaacaaatgggatcaggttcatccccgtcgggcaagcaaaattcaatccatcataagatgaatgtgagatctacctctggtcagcggagggtcgatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 17 - DNA, 1347 bp, Zea mays; identical to SEQ ID NO: 16
  CDS (1)..(1347)

SEQ ID NO: 18 - protein, 448 aa, Zea mays; identical to SEQ ID NO: 15

SEQ ID NO: 19 - DNA, 1347 bp, Zea mays; identical to SEQ ID NO: 4

SEQ ID NO: 20 - DNA, 1347 bp, Zea mays; identical to SEQ ID NO: 4
  CDS (1)..(1347)

SEQ ID NO: 21 - protein, 448 aa, Zea mays; identical to SEQ ID NO: 6

SEQ ID NO: 22 - DNA, 1347 bp, Zea mays; identical to SEQ ID NO: 4

SEQ ID NO: 23 - DNA, 1347 bp, Zea mays; identical to SEQ ID NO: 4
  CDS (1)..(1347)

SEQ ID NO: 24 - protein, 448 aa, Zea mays; identical to SEQ ID NO: 6

SEQ ID NO: 25 - DNA, 1347 bp, Zea mays; identical to SEQ ID NO: 7

SEQ ID NO: 26 - DNA, 1347 bp, Zea mays; identical to SEQ ID NO: 7
  CDS (1)..(1347)

SEQ ID NO: 27 - protein, 448 aa, Zea mays; identical to SEQ ID NO: 9

SEQ ID NO: 28 - DNA, 1347 bp, Zea mays
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcaggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 29 - DNA, 1347 bp, Zea mays; identical to SEQ ID NO: 28
  CDS (1)..(1347)

SEQ ID NO: 30 - protein, 448 aa, Zea mays
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQDIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 31 - DNA, 1078 bp, Zea mays
cgagcgattcggttgatttgatcgatttcgggtgcttcgcgattgattaggccggcaaagccgccaatccttgtgatctctcgaaggggtagagcgcggtcgaccgtcggtcatgtcgaggtgcttcccctacccgccgcctgtgtacttgggaaacccagtggccgtggccgaggcggagtcgaccgctaagcttcagaaagaaagggaaagggctcacaagaagaaagataaaaggagtgacaagaaagctccccaactgggtgagacgtccaaacattcaaagcacaaccataagaagagaaagctcgaagatgtcagcacaggtgatcaggagcccaaaaaagtattcaaagaatcagctgagctattggagaagagtggactctcagaagagcatggagctccttgttttgtacagatgtttcgtgactctcctgagagctcgcaggacagcagcaagagaagaaaggctgtcctgcccagtcccagccaagctaagaatggtaacatcattcgcatcaagctaaaaagtaaccaagatccccaatcagttcttttggagaaaccaagggttctggagcaaccactggtccaacaaatgagttcggtttcatccctgtcgagcaaacaaaattcaatcaatcgtaaggtgaatgtgagatctacagctggccagcagtgggtcaatggtgactcccaagcagtacaaaaatctttggttacagaaaccctgtcaagggcaatgcagagaactgtcccccagcctgcagtgaaggtcacacgtcgggctgatccccagctatctgttaaggcgccggttggaagatctgacctacctccaaagttttcgggaagtgtgggcccttcacctgctagagtgaccggaagattttgtcctgcacctgttaagacgcaacagagaattgagcatccaccttccatggtgtcacagagagttgatcctcaggcgaaggtgtcacagaaggaaatgggatctgctgtttgcctgccacaagctccacatcctcctgttttgcagaaacccaaggacttgcctgttcctaaacagcgggag

SEQ ID NO: 32 - DNA, 966 bp, Zea mays
  CDS (1)..(966)
atgtcgaggtgcttcccctacccgccgcctgtgtacttgggaaacccagtggccgtggccgaggcggagtcgaccgctaagcttcagaaagaaagggaaagggctcacaagaagaaagataaaaggagtgacaagaaagctccccaactgggtgagacgtccaaacattcaaagcacaaccataagaagagaaagctcgaagatgtcagcacaggtgatcaggagcccaaaaaagtattcaaagaatcagctgagctattggagaagagtggactctcagaagagcatggagctccttgttttgtacagatgtttcgtgactctcctgagagctcgcaggacagcagcaagagaagaaaggctgtcctgcccagtcccagccaagctaagaatggtaacatcattcgcatcaagctaaaaagtaaccaagatccccaatcagttcttttggagaaaccaagggttctggagcaaccactggtccaacaaatgagttcggtttcatccctgtcgagcaaacaaaattcaatcaatcgtaaggtgaatgtgagatctacagctggccagcagtgggtcaatggtgactcccaagcagtacaaaaatctttggttacagaaaccctgtcaagggcaatgcagagaactgtcccccagcctgcagtgaaggtcacacgtcgggctgatccccagctatctgttaaggcgccggttggaagatctgacctacctccaaagttttcgggaagtgtgggcccttcacctgctagagtgaccggaagattttgtcctgcacctgttaagacgcaacagagaattgagcatccaccttccatggtgtcacagagagttgatcctcaggcgaaggtgtcacagaaggaaatgggatctgctgtttgcctgccacaagctccacatcctcctgttttgcagaaacccaaggacttgcctgttcctaaacagcgggag

SEQ ID NO: 33 - protein, 322 aa, Zea mays
MSRCFPYPPPVYLGNPVAVAEAESTAKLQKERERAHKKKDKRSDKKAPQLGETSKHSKHNHKKRKLEDVSTGDQEPKKVFKESAELLEKSGLSEEHGAPCFVQMFRDSPESSQDSSKRRKAVLPSPSQAKNGNIIRIKLKSNQDPQSVLLEKPRVLEQPLVQQMSSVSSLSSKQNSINRKVNVRSTAGQQWVNGDSQAVQKSLVTETLSRAMQRTVPQPAVKVTRRADPQLSVKAPVGRSDLPPKFSGSVGPSPARVTGRFCPAPVKTQQRIEHPPSMVSQRVDPQAKVSQKEMGSAVCLPQAPHPPVLQKPKDLPVPKQRE

SEQ ID NO: 34 - DNA, 1054 bp, Zea mays
gatttcgggtgcttcgcgattgattaggccggcaaagccgccaatccttgtgatctctcgaaggggtagagcgcggtcgaccgtcggtcatgtcgaggtgcttcccctacccgccgccggtgtacttgggaaacccagtggccgtggccgaggcggagtcgaccgctaagcttcagaaagaaagggaaagggctcacaagaagaaagataaaaggagtgacaagaaagctccccaactgggtgagacgtccaaacattcaaagcacaaccataagaagagaaagcttgaagatgtcagcacaggtgatcaggagcccaaaaaagtattcaaagaatcagctgagctattggagaagagtggactctcagaagagcatggagctccttgttttgtacagatgtttcgtgactctcctgagagctcgcaggacagcagcaagagaagaaaggctgtcctgcccagtcccagccaakytaagaatggtaacatcattcgcatcaagctaaaaagtaaccaagatccccaatcagttcttttggagaaaccaagggttctggagcaaccactggtccaacaaatgagttcggyttcatccctgtcgagcaaacaaarttcaatcaatcgtaaggtgaatgkkagatctacagctggccagcagtgggtcaatggtgactcccaagcagtacaaaaatctttggttacagaaacccygtcaagggcaatgcagagaactgtcccccagcctgcagtgaaggtcacacgtcgggctgatccccagctatctgttaaggcgccggttggaagatctgacctacctccaaagttttcgggaagtgtgggcccttcacctgctagagtgaccsgaagattttgtcctscacctgttaagacgcaacagagaattsagcatccaccttccatggtgtcacagagagttgatcctcaggcgaaggtgtcacagaaggaaatgggatctgctgtttgcctgccacaagctccacatcctcctgttttgcagaaacccaaggacttgcctgttcctaaacagcggga

SEQ ID NO: 35 - DNA, 965 bp, Zea mays
  CDS (1)..(963)
atgtcgaggtgcttcccctacccgccgccggtgtacttgggaaacccagtggccgtggccgaggcggagtcgaccgctaagcttcagaaagaaagggaaagggctcacaagaagaaagataaaaggagtgacaagaaagctccccaactgggtgagacgtccaaacattcaaagcacaaccataagaagagaaagcttgaagatgtcagcacaggtgatcaggagcccaaaaaagtattcaaagaatcagctgagctattggagaagagtggactctcagaagagcatggagctccttgttttgtacagatgtttcgtgactctcctgagagctcgcaggacagcagcaagagaagaaaggctgtcctgcccagtcccagccaakytaagaatggtaacatcattcgcatcaagctaaaaagtaaccaagatccccaatcagttcttttggagaaaccaagggttctggagcaaccactggtccaacaaatgagttcggyttcatccctgtcgagcaaacaaarttcaatcaatcgtaaggtgaatgkkagatctacagctggccagcagtgggtcaatggtgactcccaagcagtacaaaaatctttggttacagaaacccygtcaagggcaatgcagagaactgtcccccagcctgcagtgaaggtcacacgtcgggctgatccccagctatctgttaaggcgccggttggaagatctgacctacctccaaagttttcgggaagtgtgggcccttcacctgctagagtgaccsgaagattttgtcctscacctgttaagacgcaacagagaattsagcatccaccttccatggtgtcacagagagttgatcctcaggcgaaggtgtcacagaaggaaatgggatctgctgtttgcctgccacaagctccacatcctcctgttttgcagaaacccaaggacttgcctgttcctaaacagcggga

SEQ ID NO: 36 - protein, 321 aa, Zea mays
  misc_feature (129)..(129): The ′Xaa′ at location 129 stands for Ala, Val, Ser, or Phe.
MSRCFPYPPPVYLGNPVAVAEAESTAKLQKERERAHKKKDKRSDKKAPQLGETSKHSKHNHKKRKLEDVSTGDQEPKKVFKESAELLEKSGLSEEHGAPCFVQMFRDSPESSQDSSKRRKAVLPSPSQXKNGNIIRIKLKSNQDPQSVLLEKPRVLEQPLVQQMSSXSSLSSKQXSINRKVNXRSTAGQQWVNGDSQAVQKSLVTETXSRAMQRTVPQPAVKVTRRADPQLSVKAPVGRSDLPPKFSGSVGPSPARVTXRFCPXPVKTQQRIXHPPSMVSQRVDPQAKVSQKEMGSAVCLPQAPHPPVLQKPKDLPVPKQR

SEQ ID NO: 37 - DNA, 446 bp, Zea mays
cagtttcagcatcaaattccccagacatgcaaaatcccgagacacctgaaaatggtcttaagagtgtgctattggaaaatcccgctgctaaaaaagatcaggtgtcattatgtccttcagttgaggatgcactggtttttactagcttaggtggaaggaaatctgaacccaaacggaatgctgataatgaaacagagataaaattggatgctcgcagtaaaggtaaatctgtcatgtcctctgtgctgcctgcttccaccacatctcatggtgcttctcataacgacctgttcatgtgccatcaatgcgcgaaaacaaactaatatatggaacaacccctacctatacttcctgtgaatccaatgggacagctcatggtagtttgcagtcgatattccctcttccacatgtagtcttccctccttgctcaccagtttccccccctt

SEQ ID NO: 38 - DNA, 23 bp, Oryza sativa
cgaaatgatggtgagaacagcat

SEQ ID NO: 39 - DNA, 22 bp, Oryza sativa
tcgactcttggcatgacttttg

SEQ ID NO: 40 - DNA, 14 bp, Oryza sativa
cagtaccgaaacaa

SEQ ID NO: 41 - DNA, 16 bp, Oryza sativa
cagtactgaaacaagg

SEQ ID NO: 42 - DNA, 20 bp, Oryza sativa
ggaacctggtgagcaattgg

SEQ ID NO: 43 - DNA, 24 bp, Oryza sativa
ggactgggtaacacaacctttctt

SEQ ID NO: 44 - DNA, 15 bp, Oryza sativa
cagacagtgcatggc

SEQ ID NO: 45 - DNA, 15 bp, Oryza sativa
cagacagagcatggc

SEQ ID NO: 46 - DNA, 25 bp, Oryza sativa
tgtcatcagtgtcatcatctggatt

SEQ ID NO: 47 - DNA, 26 bp, Oryza sativa
cccttccagtgaactttctagctatt

SEQ ID NO: 48 - DNA, 18 bp, Oryza sativa
ccgttttatgaccgtgtg

SEQ ID NO: 49 - DNA, 18 bp, Oryza sativa
ccgttttatggccgtgtg

SEQ ID NO: 50 - DNA, 21 bp, Oryza sativa
ccatttgggccactactatta

SEQ ID NO: 51 - DNA, 20 bp, Oryza sativa
tcattgtccctcctgcatcc

SEQ ID NO: 52 - DNA, 14 bp, Oryza sativa
atgctcacaactct

SEQ ID NO: 53 - DNA, 15 bp, Oryza sativa
atgctcagaactctt

SEQ ID NO: 54 - DNA, 2086 bp, Zea mays
ccattccgtctgaagaccctcctcctacctaattatttttctctgtcgtgacgtgaaatgccagcgcgcagcttcttgatccgttccggctgatcccgtggaccggacttggggttgggggaggccctctgtctccatggatgaggcagcagggctgctgttgcaagaagagggtggaggtggggaccaagaagctctcctccttccacttccacaggatgttggcctttacacaggtgatggatctgttgatgtcaaagggcgccctgcgttaaagggcactacaggcaattggaaagcatgctttttcatcctagggaatgaatgttgtgaaaggctggcctactacggaattgcaaaaaacctagttacttatttgaaagtgaagcttcatctaggcaacctcgaggctgcaagacatgttaccacttggcaagggacatgctatctcactccccttgttggaggcatcttagcagactctcgttgggggaaatactggactattgctgttttctcatcggtttactttattggcctggctattttaacgctttctgcatcagtcccagcgttgcaaccaccttcatgtttaaggacagtttgtccagaagcaagcttacttcagtatggcatattttttggtggcctctatatgattgccctagggactggaggtatcaaaccttgtgtctcctcctttggagctgatcaatttgatgacactgaccaagcagagagagctaagaagggttcattcttcaattggttctacttctgtataaatataggttcattcatatcaggcactatgatagtgtggatacaagataacactggttggggaataggctttgcgattcctactatattcatggcattagctatttcattcttcttctcagcttcaaataagtacagattccaaaaacctggtgggagtccactcacaagagtgtgccaggtggttatagcagcatttcgtaagtggcacattgaagtgccacatgatacatctctcctatatgaagttgatggccaaacttcagcaattgaaggaagccggaagctggagcacacaaatgagctcgagttccttgatagagctgctgttatctcatctgctgatctgaagagtgaatcctttaccgacccatggaagctttgcacagttacccaggtggaagaattgaagatcctaataagaatgtttcccatttgggctactactatcatattcagtgctgtttatgcccaaaactcttccatgttcatagagcagggcatggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctgaatgaaggccatctcgaccggttcttttggttaattgcagggataagctttgtaaatttgatagtttatatgggttgtgctgtcagatacagatataagaaagcctcttgattatatttatgtgaactcttgtaatgtgatttcccattcccgatatcatacttatcaaatacaatgcactgtacagatttctgaaatcctgcgattcatctgactgctttctattgcaaaacctactgtagtttattaaaaaaaaaaaaaaaaaactcgagggggggcccg

SEQ ID NO: 55 - DNA, 1779 bp, Zea mays
  CDS (1)..(1779)
atggatgaggcagcagggctgctgttgcaagaagagggtggaggtggggaccaagaagctctcctccttccacttccacaggatgttggcctttacacaggtgatggatctgttgatgtcaaagggcgccctgcgttaaagggcactacaggcaattggaaagcatgctttttcatcctagggaatgaatgttgtgaaaggctggcctactacggaattgcaaaaaacctagttacttatttgaaagtgaagcttcatctaggcaacctcgaggctgcaagacatgttaccacttggcaagggacatgctatctcactccccttgttggaggcatcttagcagactctcgttgggggaaatactggactattgctgttttctcatcggtttactttattggcctggctattttaacgctttctgcatcagtcccagcgttgcaaccaccttcatgtttaaggacagtttgtccagaagcaagcttacttcagtatggcatattttttggtggcctctatatgattgccctagggactggaggtatcaaaccttgtgtctcctcctttggagctgatcaatttgatgacactgaccaagcagagagagctaagaagggttcattcttcaattggttctacttctgtataaatataggttcattcatatcaggcactatgatagtgtggatacaagataacactggttggggaataggctttgcgattcctactatattcatggcattagctatttcattcttcttctcagcttcaaataagtacagattccaaaaacctggtgggagtccactcacaagagtgtgccaggtggttatagcagcatttcgtaagtggcacattgaagtgccacatgatacatctctcctatatgaagttgatggccaaacttcagcaattgaaggaagccggaagctggagcacacaaatgagctcgagttccttgatagagctgctgttatctcatctgctgatctgaagagtgaatcctttaccgacccatggaagctttgcacagttacccaggtggaagaattgaagatcctaataagaatgtttcccatttgggctactactatcatattcagtgctgtttatgcccaaaactcttccatgttcatagagcagggcatggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctgaatgaaggccatctcgaccggttcttttggttaattgcagggataagctttgtaaatttgatagtttatatgggttgtgctgtcagatacagatataagaaagcctcttga

SEQ ID NO: 56 - protein, 592 aa, Zea mays
MDEAAGLLLQEEGGGGDQEALLLPLPQDVGLYTGDGSVDVKGRPALKGTTGNWKACFFILGNECCERLAYYGIAKNLVTYLKVKLHLGNLEAARHVTTWQGTCYLTPLVGGILADSRWGKYWTIAVFSSVYFIGLAILTLSASVPALQPPSCLRTVCPEASLLQYGIFFGGLYMIALGTGGIKPCVSSFGADQFDDTDQAERAKKGSFFNWFYFCINIGSFISGTMIVWIQDNTGWGIGFAIPTIFMALAISFFFSASNKYRFQKPGGSPLTRVCQVVIAAFRKWHIEVPHDTSLLYEVDGQTSAIEGSRKLEHTNELEFLDRAAVISSADLKSESFTDPWKLCTVTQVEELKILIRMFPIWATTIIFSAVYAQNSSMFIEQGMVLDKRIGSFNIPPASLSTFDVISVIMWVPLYDRILVPLARKFTGREKGFSELQRMGIGLVLSILAMVSAALVELKRLEIARSEGLIHEKAAVPMSILWQIPQYFLVGAAEVFTCIGQVEFFYDQAPDAMRSLCSALALITVSLGNYISSIILTLVSYITTQGGDPGWIPDNLNEGHLDRFFWLIAGISFVNLIVYMGCAVRYRYKKAS

SEQ ID NO: 57 - DNA, 499 bp, Sorghum bicolor
ttgcagaagagggtggagggtagggaccacgaaccgctcctccttccacttccacaggatgctggcctttacacaggtgatggatctgttgatgtcaaagggtgtcctgcattaaagggcactacaggcaattggaaagcatgttttttcatcctagggaatgagtgttgtgaaaggctggcctactacggaattgcaaaaaacctagttacttatttgaaagtgaagcttcatctaggcaaccttgaggctgcaagacatgttaccacttggcaagggacatgctatcttactccccttgttggagccatcttagcagattctcattgggggaaatactggacaattgctgttttctcatcagtttactttattggcctggctattttgacgctgtcagcatcagtcccagcattgcagccaccttcatgtttaagaacagtttgtccagaagcaagcttacttcagtatggcgtattttttgttggtctctatatga

SEQ ID NO: 58 - DNA, 604 bp, Sorghum bicolor
  CDS (1)..(525)
attctggtgccagtagctagaaaattcactggaagggagaagggtttttctgagctccagcggatgggaattggattagccctgtcaattcttgcgatggtatctgcagctcttgttgagttgaagcgtttagagattgccaggtctgaaggtcttattcatgagaaggctgctgttccaatgagcattctttggcaaataccacaatatttcttggtgggtgctgctgaggtgtttacttgtataggtcaagtcgagttcttttacgatgaggccccagatgccatgaggagtttatgtagtgcatttgcacttattacagtctcactgggaagctatataagctccatcatactgacgttggtgtcgtgcattacaactcagggaggagatcctggatggatccctgacaatctgaatgaaggccatctcgaccggttcttttggttaattgctgggataagctttgtgaatttgatagtttacatgggctgtgctgtgagatacagatataagaaagcctcttgattatatttatgtgaactcttgtaatgtgattccaggtgccatacttatcaaatacattgcattgtmcagacttctg

SEQ ID NO: 59 - protein, 175 aa, Sorghum bicolor
ILVPVARKFTGREKGFSELQRMGIGLALSILAMVSAALVELKRLEIARSEGLIHEKAAVPMSILWQIPQYFLVGAAEVFTCIGQVEFFYDEAPDAMRSLCSAFALITVSLGSYISSIILTLVSCITTQGGDPGWIPDNLNEGHLDRFFWLIAGISFVNLIVYMGCAVRYRYKKAS

SEQ ID NO: 60 - DNA, 1130 bp, Saccharum officinarum
ccgtggaccgtggactggactaggggttgggggaggccctctgtctccaactctccatggatgaggcagcaggactgctgttgcaagaagagggtggaggtgaggaccacgaaccgctcctccttccacttccacaggacgctggcctttacacaggtgatggatctgttgatgtcaaagggcgccctgcattaaagcgcactacaggcaattggaaagcatgttttttcatcctagggaatgagtgttgtgaaaggttggcctactacggaattgcgaaaaacctagttacttatttgaaagtgaagcttcatctaggcaacctcgaggctgcaagacatgtcaccacttggcaagggacatgctatctcactccccttgttggagccatcttagcagattctcattgggggaaatactggacaattgctgttttctcatcagtttactttattggcctggctattttgacgctgtcagcatcagtcccagcattgcagccaccttcatgtttaagaacagtttgtccagaagcaagcatacttcagtatggcatattttttgttggtctctatatgatagccctagggactggaggtatcaaaccttgtgtctcctcctttggagctgatcaatttgatgacactgacccagcagagagagctaagaagggttccttcttcaattggttctacttctgtataaatattggttcattcatatcaggcactatcatagtgtggatacaagataacactggttggggaataggatttgcgattcctactatattcatggcattagctatttcattcttcttacaagcttcaaataagtacagattccaaaaacctggtgggagttcactcataagagtgtgtcaggtagttatagcagcatttcgtaagtggcatatagaagtgccacatgatacatctttcctttatgaagttgatggccaaacttcaacaattgagggaagcccgaagctggaacacacaaatgagctcgagttcctggatagagctggcgttattttatttggtgatctgaagagcgaattccttacaacccattgaaactttgcacaattccccagttggaagaattgaagattctaataagaatgt

SEQ ID NO: 61 - DNA, 595 bp, Saccharum officinarum
  CDS (1)..(594)
atggatgaggcagcaggactgctgttgcaagaagagggtggaggtgaggaccacgaaccgctcctccttccacttccacaggacgctggcctttacacaggtgatggatctgttgatgtcaaagggcgccctgcattaaagcgcactacaggcaattggaaagcatgttttttcatcctagggaatgagtgttgtgaaaggttggcctactacggaattgcgaaaaacctagttacttatttgaaagtgaagcttcatctaggcaacctcgaggctgcaagacatgtcaccacttggcaagggacatgctatctcactccccttgttggagccatcttagcagattctcattgggggaaatactggacaattgctgttttctcatcagtttactttattggcctggctattttgacgctgtcagcatcagtcccagcattgcagccaccttcatgtttaagaacagtttgtccagaagcaagcatacttcagtatggcatattttttgttggtctctatatgatagccctagggactggaggtatcaaaccttgtgtctcctcctttggagctgatcaatttgatgacactgacc

SEQ ID NO: 62 - protein, 198 aa, Saccharum officinarum
MDEAAGLLLQEEGGGEDHEPLLLPLPQDAGLYTGDGSVDVKGRPALKRTTGNWKACFFILGNECCERLAYYGIAKNLVTYLKVKLHLGNLEAARHVTTWQGTCYLTPLVGAILADSHWGKYWTIAVFSSVYFIGLAILTLSASVPALQPPSCLRTVCPEASILQYGIFFVGLYMIALGTGGIKPCVSSFGADQFDDTD

SEQ ID NO: 63 - DNA, 817 bp, Saccharum officinarum
  CDS (1)..(816)
gatggccaaacttcagcaattgagggaagccggaagctggagcacacaaatgagctcgagttccttgatagagctgccgttatctcatctgctgatctgaagagcgaatcctttacagacccatggaagctttgctcagttacccaggtggaagaattgaagatcctaataagaatgtttcccatttgggctactactatcatattcagtgctgtttrcgcccaaaactcttccatgttcatagagcagggcatggttcttgacaagcgcattggatctttcaacattcctcctgcatctctctcaacttttgatgtaatcagcgtcatcatattggttccactttatgaccgcattctggtgccaatagctagaaaattcaccggaagggagaagggtttttcggagctacagcggatgggaattggattagtcctgtcaattattgcgatggtatctgcagctcttgttgagttgaagcgtttagagattgccaggtctgaaggtcttattcatgagaaggctgctgttccaatgagcattctttggcaaataccacaatatttcttggtgggtgctgctgaggtgtttacttgtataggccaagctgagttcttttacgatcaggccccagatgccatgaggagtttatgtagtgcatttgcacttattacagtctcactgggaagctatataagctccatcatactgacgttggtggcgtacattacagctcaaggaggagatcctggatggatccctgacaatctgaatgaaggccatctcgaccggttcttttggttaattgcaaggataa

SEQ ID NO: 64 - protein, 272 aa, Saccharum officinarum
  misc_feature (73)..(73): The ′Xaa′ at location 73 stands for Cys, or Tyr.
DGQTSAIEGSRKLEHTNELEFLDRAAVISSADLKSESFTDPWKLCSVTQVEELKILIRMFPIWATTIIFSAVXAQNSSMFIEQGMVLDKRIGSFNIPPASLSTFDVISVIILVPLYDRILVPIARKFTGREKGFSELQRMGIGLVLSIIAMVSAALVELKRLEIARSEGLIHEKAAVPMSILWQIPQYFLVGAAEVFTCIGQAEFFYDQAPDAMRSLCSAFALITVSLGSYISSIILTLVAYITAQGGDPGWIPDNLNEGHLDRFFWLIARI

SEQ ID NO: 65 - DNA, 675 bp, Triticum aestivum
  CDS (3)..(596)
tcagtgttcctcctgcgtccctctcgagctttgacgtaatcagtgtcatgatctgggttccgctttatgaccgtgttctcatacctatagccagaaagttcactggaagggaaaagggtttctcagaactacaacggattggcattggattggtgctgtccattattgcaatggtgtctgcagcttttgttgagttgaagcgcttggagattgccacatctgaaggtcttatccatgagaagtctgcggttccaatgagcattctttggcaaataccacagtatttccttgttggcgctgccgaggttttcactaatataggtctacttgagttttcgtacgatcaggcaccagatgccatgaggagtttatgtactgcatttgcgctcgtcatggtctcagcggggagctatttaagctcattcatattgaccctggtgtcgtatgttacaactcgaggtggagatcctggatggatcccggataacatgaatgaaggccatcttgaccggttcttttggttgattgcagggatcagctttgtgaatttgctggtttacatcagttgtgcgatgaaatacaaatataagaatgtgtgatggtttctccaaaaaattgtgtgatggtcatacctactgtggaataagttcttgtaatttcagattccatattcat

SEQ ID NO: 66 - protein, 198 aa, Triticum aestivum
SVPPASLSSFDVISVMIWVPLYDRVLIPIARKFTGREKGFSELQRIGIGLVLSIIAMVSAAFVELKRLEIATSEGLIHEKSAVPMSILWQIPQYFLVGAAEVFTNIGLLEFSYDQAPDAMRSLCTAFALVMVSAGSYLSSFILTLVSYVTTRGGDPGWIPDNMNEGHLDRFFWLIAGISFVNLLVYISCAMKYKYKNV

SEQ ID NO: 67 - DNA, 675 bp, Triticum aestivum
  CDS (3)..(596)
tcagtgttcctcctgcgtccctctcgagctttgacgtgatcagtgtcatgatctgggttccactttatgaccgtgttctcatacctatagccagaaagttcactggaagagaaaagggtytctcggaactacaacggattggcattggattggtgctgtccattattgcaatggtgtctgcagcttttgttgagttgaagcgcttggagattgccgcgtctgaaggtcttatccatgagaaggctgtggttccgatgagcattctttggcaaataccgcagtatttctttgttggtgctgccgaggttttcactaatataggtcagcttgagttcttctatgatcaggccccagatgccatgaggagtttatgtgctgcatttgcgctcgtcacggtctcagcggggagctatttaagctcgttcatactgaccatggtgtcgtatgttacaactcgaggtggagrtcctggatggatcccggataacctgaatgaaggccatcttgaccggttcttctggttgattgcagggatcagctttgtgaatttgctggtttacatcagttgtgcgatgaaatacaaatataagaatgtgtgatggtttctccaaaaaaatgtgtgatgggcatacctactgtggaataagttcttgtgatttcagattccatattcat

SEQ ID NO: 68 - protein, 198 aa, Triticum aestivum
  misc_feature (40)..(40): The ′Xaa′ at location 40 stands for Leu, or Phe.
SVPPASLSSFDVISVMIWVPLYDRVLIPIARKFTGREKGXSELQRIGIGLVLSIIAMVSAAFVELKRLEIAASEGLIHEKAVVPMSILWQIPQYFFVGAAEVFTNIGQLEFFYDQAPDAMRSLCAAFALVTVSAGSYLSSFILTMVSYVTTRGGXPGWIPDNLNEGHLDRFFWLIAGISFVNLLVYISCAMKYKYKNV

SEQ ID NO: 69 - DNA, 675 bp, Triticum aestivum
  CDS (3)..(596)
tcagtgttcctcctgcgtccctctcgagttttgacgtaatcagtgtcatgatctgggttccactttatgaccgtgttctcatacctatagccagaaagttcactggaagggaaaagggtttctcggaactacaacggattggcattggattggtgctgtccattattgcaatggtgtctgcagcttttgttgagttgaagcgcttggagattgccgcgtctgaaggtcttatccatgagaaggctgtggttccgatgagcattctttggcaaataccgcagtatttctttgttggtgctgccgaggttttcactaatataggtcagcttgagttcttctatgatcaggccccagatgccatgaggagtttatgtgctgcatttgcgctcgtcacggtctcagcggggagctatttaagctcgttcatactgaccatggtgtcgtatgttacaactcgaggtggagatcctggatggatcccggataacctgaatgaaggccatcttgaccggttcttctggttgattgcagggatcagctttgtgaatttgctggtttacatcagttgtgcgatgaaatacaaatataagaatgtgtgatggtttctccaaaaaaatgtgtgatgggcatacctactgtggaataagttcttgtgatttcagattccatattcat

SEQ ID NO: 70 - protein, 198 aa, Triticum aestivum
SVPPASLSSFDVISVMIWVPLYDRVLIPIARKFTGREKGFSELQRIGIGLVLSIIAMVSAAFVELKRLEIAASEGLIHEKAVVPMSILWQIPQYFFVGAAEVFTNIGQLEFFYDQAPDAMRSLCAAFALVTVSAGSYLSSFILTMVSYVTTRGGDPGWIPDNLNEGHLDRFFWLIAGISFVNLLVYISCAMKYKYKNV

SEQ ID NO: 71 - DNA, 1522 bp, Hordeum vulgare
  CDS (3)..(932)
  misc_feature (1498)..(1498): n is a, c, g, or t
atcttgaagctgcaagaaatgttatcacttggcaagggacatgctatctgacttccctcgttggagccatcctagcagattcttattggggaaagtactggactattgttgttttctcgtcgatttatttcattggtctggctggtttaacgctttcagcatcacttccagcacttcaaccaccttcatgttcaggatctgtttgcccagaaccaagcctacttcagaatggcacatttttcctgggcctctatatgattgccctaggaaccggaggcattaaaccttgtgtgtcatcctttggagctgaccaatttgatgtcagtgatccgacagagagagtaaagcagggttccttcttcaattggttctatttctgcataaatgtcggtgcactcttatcaggcactgttattgtttggatacaagataactcaggttggggaataggatttgccattcctactgtatttatggcactggctatcgcaagcttcttttcagcctcaaatatgtatagatttcagaaacccggtgggagtccaattacaagagtgtgccaggttgttgtcgcagcattccgtaagtggcatatcgagttgccactygacgcttctcttctgtatgaagttgatggtcgaaagtcagcaatagagggaagccgaaagctggagcacacaagtgaacttgaattccttgacaaggctgctatcatctcatctactgatgccaagagtgacttttctgcaaacccatggaggctatgcactgtcacccaagtggaagaactgaagatcctagtaagaatgttcccagtttgggcgactacwatcatattcagcggggtatttgctcagaactccgtattcgtggagcagggaatggttcttgacaaacgggttggatctttcgatattcctctgcatccctattaactttcgacgtaatcagtgtcatgatctggattccaatttatgaccgtatactcatacccatagctagaaagttcactggaagggaaaagggtttctctgagctacagcgaatgggcattggattagtcctatccattatgacaatggtatctgcagctcttgttgagttgaagcgcttagagattgccaggactgagggtcttgttcatgagaatgttgctgttccgatgagcattctttggcaaataccacagtattgctttgctggtgctgccgaggttttcaccgctataggtcaagtcgagttcttctatggtcaggccccagatgccatgaggagcttatgtgctgcattggcacttgttacggtcacggtgggaagctatttaagctcaatcatattgaccttggtgtcataccttacaactcaaggaggagatgcaggatggatcccagataacttgaatgaaggccatctcgaccggtttttttggttgctggcagggatcagctttgtaaatttgctggtttacattggttgcgcaatgagatacaaatataanaatgtgtgatggttatagttactg

SEQ ID NO: 72 - protein, 310 aa, Hordeum vulgare
  misc_feature (202)..(202): The ′Xaa′ at location 202 stands for Leu.
LEAARNVITWQGTCYLTSLVGAILADSYWGKYWTIVVFSSIYFIGLAGLTLSASLPALQPPSCSGSVCPEPSLLQNGTFFLGLYMIALGTGGIKPCVSSFGADQFDVSDPTERVKQGSFFNWFYFCINVGALLSGTVIVWIQDNSGWGIGFAIPTVFMALAIASFFSASNMYRFQKPGGSPITRVCQVVVAAFRKWHIELPXDASLLYEVDGRKSAIEGSRKLEHTSELEFLDKAAIISSTDAKSDFSANPWRLCTVTQVEELKILVRMFPVWATXIIFSGVFAQNSVFVEQGMVLDKRVGSFDIPLHPY

SEQ ID NO: 73 - DNA, 1661 bp, Hordeum vulgare
  CDS (3)..(932)
atcttgaagctgcaagaaatgttaccacttggcaagggacatgctatctgtctcccctcattggagccatcctagcagattcttattggggaaagtactggactattgctgttttctcatcaatttatttcatcggcctgtctgttttaactctttcagcatcgcttccagcacttcagccaccttcatgtttaggaaccgtttgtccagaagcaagcttacttcaaaatggcacatttttcctaggtctctatatgattgccctagggaccggaggtattaaaccatgtgtgtcatcctttggggctgatcaatttgatgacagtgatccgacagagagagtaaagcagggttccttcttcaactggttctatttctgcataaatatcggtgcattcatatcaggcactgttattgtttggatacaagataactcgggttggggaataggatttgccattcctactgtatttatggcattggctattgcaagcttcttttcagcttcagatatgtatagatttcagaaacctggtgggagtccacttacaagagtgtgccaggtcgttgtcgcagcgttccgtaagtggcatgttgaactgccacatgacactgctcttttatatgaagttgataatcaaaattcagcaatagatggaagcagaaagctagagcacacaagtgaacttgaattccttgacaaggctgccatcatctcatctactgatgccaagagtgacctccttacaaacccatggaggctttgcacggtcacccaggtggaagaactaaagatcctagtaagaatgttcccggtctgggctactactatcatattcaatgcggtgtacgctcagaactcttccatgttcttagagcagggaatggttctcgacaagcgggttgggtctttcaatgttcctcctgcgtccctctcgagctttgacgtaatcagtgtcatgatctgggttccactttatgaccgtgttctcatacctatagctaggaagttcactggaagggaaaagggtttctcagagctacaacggattggcattggactagtgctttccatttttgcaatggtgtctgcagcttttgtcgaggtgaagcgcttggagattgccaggtccgaaggtcttatccatgagaaggctgcggttccgatgagcattctttggcaaataccgcagtatttctttgttggcgctgccgaggttttcactaatataggtcagcttgagttcttctatgatcaggccccagatgccatgagaagtttatgtgctgcatttgcactcgtcacggtctcagcagggagctatttaagctcattcatattgaccttggtgtcttacgttacaactcgaggtggagatcctggatggatcccggataacctgaacgaaggccatcttgaccggttcttttggttgattgcaggggtcagctttgtgaatttgctggtttacatcagttgtgcaatgaaatacaaatataagaatgtgtgatggtttatctccacaaaaatgtgtgatggtcatacctgctgtggaatcagtccttgtaatctcagattccaatatccatgaaagctgtgcattttatagactaagacatcacatgagcyggccctcctcaacaggtgcctcaagaaaaag

SEQ ID NO: 74 - protein, 310 aa, Hordeum vulgare
LEAARNVTTWQGTCYLSPLIGAILADSYWGKYWTIAVFSSIYFIGLSVLTLSASLPALQPPSCLGTVCPEASLLQNGTFFLGLYMIALGTGGIKPCVSSFGADQFDDSDPTERVKQGSFFNWFYFCINIGAFISGTVIVWIQDNSGWGIGFAIPTVFMALAIASFFSASDMYRFQKPGGSPLTRVCQVVVAAFRKWHVELPHDTALLYEVDNQNSAIDGSRKLEHTSELEFLDKAAIISSTDAKSDLLTNPWRLCTVTQVEELKILVRMFPVWATTIIFNAVYAQNSSMFLEQGMVLDKRVGSFNVPPAS

SEQ ID NO: 75 - DNA, 1344 bp, Hordeum vulgare
  CDS (1)..(1344)
atgtcgaggtgcttcccctacccgccgccggggtacgtgcgaaacccagtggtggccgtggccgcggccgaagcgcaggcgaccactaagctccagaaagaaagggaaaaggctgaaaagaagaaagagaaaaggagtgacaggaaagctcttccacatggtgagatatccaagcattcaaagcgaacccaccacaagaagagaaaacatgaagacatcaataatgctgatcagaagtcccggaaggtttcctccatggaacctggtgagcaattggagaagagtggactctcagaagagcatggagctccttgctttactcagacagagcatggctctccagagagttcacaggacagcagcaagagaagaaaggttgtgttacccagtcctagccaagctaagaatggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtaccgaaacaaggtatgccaaccccagcaaaagtcatgccaagagtcgatgttcctccatctatgagggcatcaaaggaaaggattggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaaggtctcccagacagagatcaagccggaagtacagcccccaattctgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatctcggggaccaggattggctgcttgatagtacgaggaaacctgatgctggcattggcaactgcagagaaattgttgatccacttacttctcaatcagcagagcagttctcattgcagcctagggcgattcatttaccagaccttcatgtctatcagttgccatatgtggttccattctag

SEQ ID NO: 76 - protein, 447 aa, Hordeum vulgare
MSRCFPYPPPGYVRNPVVAVAAAEAQATTKLQKEREKAEKKKEKRSDRKALPHGEISKHSKRTHHKKRKHEDINNADQKSRKVSSMEPGEQLEKSGLSEEHGAPCFTQTEHGSPESSQDSSKRRKVVLPSPSQAKNGNILRIKIRRDQDSSASLSEKSNVVQTPVHQMGSVSSLPSKKNSMQPHNTEMMVRTASTQQQSIKGDFQAVPKQGMPTPAKVMPRVDVPPSMRASKERIGLRPAEMLANVGPSPSKAKQIVNPAAAKVTQRVDPPPAKASQRIDPLLPSKVHIDATRSFTKVSQTEIKPEVQPPILKVPVAMPTINRQQIDTSQPKEEPCSSGRNAEAASVSVEKQSKSDRKKSRKAEKKEKKFKDLFVTWDPPSMEMDDMDLGDQDWLLDSTRKPDAGIGNCREIVDPLTSQSAEQFSLQPRAIHLPDLHVYQLPYVVPF

SEQ ID NO: 77 - DNA, 329 bp, Triticum aestivum
  CDS (1)..(177)
  misc_feature (18)..(18): n is a, c, g, or t
accgcaccggattggttntcggcggtcaagaaacctgatgcctcaacagcagctgcaaggcaagtgatggtttggcgcccatggagctggagttttcatggcaaccaagggctattcatttgcctgaccttcatatgtatcagttgccatatgttgttcccttttaggtttgtgtaggaagactgaggttagatgagaagtagaagagatgttgggagatagctgtgccggtgtgggagatagtgttcccccacggcagttttccagctttgtttcctagtttttcttttccaggacgatggatttggcaaccttctgtagtgttgtta

SEQ ID NO: 78 - protein, 58 aa, Triticum aestivum
  misc_feature (6)..(6): The ′Xaa′ at location 6 stands for Leu, or Phe.
TAPDWXSAVKKPDASTAAARQVMVWRPWSWSFHGNQGLFICLTFICISCHMLFPFRFV

SEQ ID NO: 79 - DNA, 1353 bp, Sorghum bicolor
  CDS (1)..(1353)
atgtcgaggtgcttcccctacccgccgccggggtacgtgcgaaacccagtggccgtggccgtggccgaggcggagtcgaccgctaagctccagaaagaaagggaaaaggccgaaaagaagaaagagaaaaggagtgacaagaaagctccccagaagggtgagacgtctaaacattcaaagcacagccataagaagagaaagcttgaagatgtcatcaaagttgggcaggatcccaaaagggaatccaaagaatcagttgagcagttggagaagagtggactctcagaagagcatggagctccttgttttgtacagacgatccgcgactctcctgagagttcacaggacagcagcaagaggcgaaaggttgtcctgcccagtcctagccaagctaagaatgggaacatccttcgcatcaagattaaaagtaatcaagattcccaatcagctctttcggagaaaccaatggttcctgagcagccattggtccgacaaatgggatcaggctcgtccctgttgggcaagcaaaattctatccatcataaggtgaatgtgagatctacatctgctcagcagagggtaactggtgactcccaagcagtacaaaaatgtattattacagaaagtctggcaaagaccatgcagagagttgttccccagcctgcagcgaaggtcacacatccggttcatcccccgttgtctgttaaggcgccagttggaagatctgacctacctccaaagttttcgggaagtgtggcgtcttcacctgctggagtgatgggaagatttgatcctccacctgttaagatggtgtcacagggagttcagcgtccagcttccatggtgtcacagaaagttgatcctcagttagcgaaggtgttacagaaagaaacgagatctgctgtttgcctaccagacgccccgcagcctcctgttctgcaaaaacccaaggacttgactgttctcaagcagcaggatctcatcacctctttgccaaaagaagagccttgcttctctggtagaagtgcagaaacagttcaagtgcaggatactaagctctcccggtcagatcggaagaaaatccgcaaagctgagaagaaagaaaagaagttcagagatctgtttgttacctggaatccgatatcgctagagaatgaaggttcagatcttggtgatcaagattggctgttgagcagtacaaggaactctgatgctagcatggctcaatgcaaatcaactggtggtgtagggccgatccatccaatggtgcagcagcagccttctttgcaacccagggccacttttctgccggaccttcatatgtaccagctgccatatgtcgtaccattttaa

SEQ ID NO: 80 - protein, 450 aa, Sorghum bicolor
MSRCFPYPPPGYVRNPVAVAVAEAESTAKLQKEREKAEKKKEKRSDKKAPQKGETSKHSKHSHKKRKLEDVIKVGQDPKRESKESVEQLEKSGLSEEHGAPCFVQTIRDSPESSQDSSKRRKVVLPSPSQAKNGNILRIKIKSNQDSQSALSEKPMVPEQPLVRQMGSGSSLLGKQNSIHHKVNVRSTSAQQRVTGDSQAVQKCIITESLAKTMQRVVPQPAAKVTHPVHPPLSVKAPVGRSDLPPKFSGSVASSPAGVMGRFDPPPVKMVSQGVQRPASMVSQKVDPQLAKVLQKETRSAVCLPDAPQPPVLQKPKDLTVLKQQDLITSLPKEEPCFSGRSAETVQVQDTKLSRSDRKKIRKAEKKEKKFRDLFVTWNPISLENEGSDLGDQDWLLSSTRNSDASMAQCKSTGGVGPIHPMVQQQPSLQPRATFLPDLHMYQLPYVVPF

(19) United States
(12) Patent Application Publication
(10) Pub. No.: US 2011/0083229 A1
(43) Pub. Date: Apr. 7, 2011

(54) EG1117 And EG307 Polynucleotides And Uses Thereof

(75) Inventor: Walter Messier, Longmont, CO (US)

(73) Assignee: EVOLUTIONARY GENOMICS, INC., Lafayette, CO (US)

(21) Appl. No.: 12/795,584

(22) Filed: Dec. 10, 2010

Related U.S. Application Data
(63) Continuation of application No. 11/394,367, filed on Mar. 29, 2006, now abandoned.
(60) Provisional application No. 60/666,511, filed on Mar. 29, 2005.
(60) Provisional application No. 60/774,939, filed on Feb. 17, 2006.

(51) Int. Cl. A01H 1/02; C12Q 1/68; C07H 21/04; C07K 14/415; C12N 5/10; A01H 5/00

(52) U.S. Cl. 800/266; 435/6; 536/23.6; 530/370; 435/419; 800/298

(57) ABSTRACT

The present invention provides methods for identifying polynucleotide and polypeptide sequences which may be associated with a commercially relevant trait in plants, specifically, so-identified polynucleotides and polypeptide sequences for yield-related genes EG307 and EG1117 for corn, wheat, barley, sorghum, and sugarcane. Sequences thus identified are useful in enhancing commercially desired traits in domesticated plants or wild ancestor plants, identifying related polynucleotide sequences, genotyping a plant, and marker assisted breeding. Sequences thus identified may also be used to generate heterologous DNA, transgenic plants, and transfected host cells.

### CROSS REFERENCE TO RELATED APPLICATIONS

The present application is a continuation of U.S. application Ser. No. 11/394,367, entitled “EG1117 And EG307 Polynucleotides And Uses Thereof,” filed Mar. 29, 2006, now abandoned, which is a non-provisional of U.S. Ser. No. 60/666,511, entitled “EG1117 And EG307 Polynucleotides And Uses Thereof,” filed Mar. 29, 2005, now expired, and is also a non-provisional of U.S. Ser. No. 60/774,939, entitled “Yield-related Polynucleotides and Polypeptides in Crop Plants,” filed Feb. 17, 2006, now expired; each of which is incorporated by reference herein in their entireties.

### FIELD OF THE INVENTION

The invention relates to molecular and evolutionary techniques to identify polynucleotide and polypeptide sequences corresponding to commercially relevant traits, such as yield, in ancestral and domesticated plants, the identified polynucleotide and polypeptide sequences, and methods of using the identified polynucleotide and polypeptide sequences.

### BACKGROUND OF THE INVENTION

Humans have bred plants and animals for thousands of years, selecting for certain commercially valuable and/or aesthetic traits. Domesticated plants differ from their wild ancestor or family members in such traits as yield, short day length flowering, protein and/or oil content, ease of harvest, taste, disease resistance and drought resistance. Domesticated animals differ from their wild ancestor or family members in such traits as fat and/or protein content, milk production, docility, fecundity and time to maturity. At the present time, most genes underlying the above differences are not known, nor, as importantly, are the specific changes that have evolved in these genes to provide these capabilities. Understanding the basis of these differences between domesticated plants and animals and their wild ancestor or family members will provide useful information for maintaining and enhancing those traits. In the case of crop plants, identification of the specific genes that control desired traits will allow direct and rapid improvement in a manner not previously possible.

The identification in domesticated species of genes that have evolved to confer unique, enhanced or altered functions compared to homologous ancestral genes could be used to develop agents to modulate these functions. The identification of the underlying domesticated species genes and the specific nucleotide changes that have evolved, and the further characterization of the physical and biochemical changes in the proteins encoded by these evolved genes, could provide valuable information on the mechanisms underlying the desired trait. This valuable information could be applied to DNA marker assisted breeding or DNA marker assisted selection. Alternatively, this information could be used in developing agents that further enhance the function of the target proteins. Alternatively, further engineering of the responsible genes could modify or augment the desired trait. Additionally, the identified genes may be found to play a role in controlling traits of interest in other domesticated plants.

Humans, through artificial selection, have provided intense selection pressures on crop plants. This pressure is reflected in evolutionarily significant changes between homologous genes of domesticated organisms and their wild ancestor or family members. It has been found that only a few genes, e.g., 10-15 per species, control traits of commercial interest in domesticated crop plants. These few genes have been exceedingly difficult to identify through standard methods of plant molecular biology.

Methods for identifying genes changed due to domestication are described in related patents and applications listed above. Methods for DNA marker assisted breeding (MAB) and DNA marker assisted selection (MAS) are well known to those skilled in the art and have been described in many publications (see for example Peleman and van der Voort, Breeding by Design, TRENDS in Plant Science 8(7):330-334). Such methods can make plant breeding more efficient by increasing the ability to select and incorporate specific alleles associated with a desired phenotype during the development of new plant varieties. One problem with markers generally used today is that they can become separated from target genes or traits through recombination (see Holland in Proceedings of the 4^{th} International Crop Science Congress 26 Sep.-1 Oct. 2004, Brisbane, Australia). In fact, Holland cites examples where use of markers was better than conventional breeding, and other examples where conventional breeding gave better results than marker assisted breeding. Holland states that “it is not likely that markers will soon be generally useful for manipulating complex traits like yield”. What is needed for markers to be useful for manipulating complex traits like yield are the specific genes underlying such complex traits instead of markers that are only sometimes associated with such complex traits.

All patents and applications referred to herein are incorporated by reference herein in their entirety, including U.S. Application Ser. No. 60/349,088, filed Jan. 16, 2002, entitled “Methods to Identify Evolutionarily Significant Changes in Polynucleotide and Polypeptide Sequences in Domesticated Plants and Animals;” U.S. Application Ser. No. 60/349,661, filed Jan. 17, 2002, entitled “Validation of Agriculturally Important Gene Candidates Selected by an Adapted-Traits Discovery Platform;” U.S. Application Ser. No. 60/349,727, filed Jan. 17, 2002, entitled “Computational Platform for the Engineering of Precise Transgene Regulation;” U.S. application Ser. No. 10/522,393, filed Jan. 25, 2005, entitled “Detection Of Evolutionary Bottlenecking By DNA Sequencing As A Method To Discover Genes Of Agronomic Value;” U.S. Ser. No. 60/402,340, filed Aug. 8, 2002, entitled “Detection Of Evolutionary Bottlenecking By DNA Sequencing As A Method To Discover Genes Of Agronomic Value;” U.S. Application Ser. No. 60/666,511, filed Mar. 29, 2005 entitled “Yield-Related Polynucleotides and Polypeptides in Crop Plants;” U.S. Application Ser. No. 60/714,142, filed Sep. 2, 2005, entitled “Yield-Related Polynucleotides And Polypeptides In Crop Plants;” and U.S. application Ser. No. 10/345,820, filed Jan. 16, 2003, entitled “EG1117 Polynucleotides And Uses Thereof;” which is a nonprovisional of U.S. Application Ser. No. 60/368,541, filed Mar. 29, 2002, entitled “Methods to Identify Evolutionarily Significant Changes in Polynucleotide and Polypeptide Sequences in Domesticated Plants and Animals;” U.S. application Ser. No. 10/079,042, filed Feb. 19, 2002 (WO 03/062382) which is a continuation-in-part of 09/875,666, filed Jun. 6, 2001, which is a continuation of U.S. application Ser. No. 09/368,810, filed Aug. 3, 1999, now U.S. Pat. No. 6,274,319, which is a continuation-in-part of U.S. application Ser. No. 09/240,915, filed Jan. 29, 1999, now U.S. Pat. No. 6,228,586, this application also claims the benefit of U.S. Application Ser. No. 60/666,511 and U.S. Application Ser. No. 60/774,639.

### DESCRIPTION OF THE FIGURES

FIG. 1 shows 1000-Grain weight vs. allele type (data from Table III), showing data in from Table III showing the range of grain weights correlated with either the ancestral (square) or derived (triangle) allele for either EG307 or EG1117.

FIG. 2 shows single factor additive model corrected for line effects showing effects of allele of EG307 or EG 1117 on phenotypic traits (>1 SD or <−1 SD indicates a major gene effect); phenotypic data converted to Z scores, values expressing to what extent a trait is affected by a particular genotype.

### DETAILED DESCRIPTION OF THE INVENTION

With the present invention, the inventors have identified genes, polynucleotides, and polypeptides corresponding to EG1117 (for Z. mays mays (corn), S. bicolor (sorghum), S. officinarum (sugarcane), T. aestivum (wheat), H. vulgare (barley), and O. sativa (domesticated rice)), EG307 (elite corn alleles, non-elite corn alleles, T. aestivum, H. vulgare, S. bicolor, and O. sativa). The polynucleotides and polypeptides of the present invention are useful in a variety of methods such as a method to identify a polynucleotide sequence that is associated with yield in a plant; a method of determining whether a plant has one or more of a polynucleotide sequence comprising an EG1117 or EG307 sequence; and a method for marker assisted breeding of plants for a particular EG1117 or EG307 sequence. The polynucleotides and polypeptides of the present invention are also useful for creating plant cells, propagation materials, transgenic plants, and transfected host cells.

Additionally, the polynucleotides and polypeptides of the present invention may be used as markers for improved marker assisted selection or marker assisted breeding. Moreover, such polynucleotides and polypeptides can be used to identify homologous genes in other species that share a common ancestor or family member, for use as markers in breeding such other species. For example, maize, rice, wheat, millet, sorghum and other cereals share a common ancestor or family member, and genes identified in rice can lead directly to homologous genes in these other grasses. Likewise, tomatoes and potatoes share a common ancestor or family member, and genes identified in tomatoes by the subject method are expected to have homologues in potatoes, and vice versa.

The practice of the present invention employs, unless otherwise indicated, conventional techniques of molecular biology, genetics and molecular evolution, which are within the skill of the art. Such techniques are explained fully in the literature, such as: “Molecular Cloning: A Laboratory Manual”, second edition (Sambrook et al., 1989); “Oligonucleotide Synthesis” (M. J. Gait, ed., 1984); “Current Protocols in Molecular Biology” (F. M. Ausubel et al., eds., 1987); “PCR: The Polymerase Chain Reaction”, (Mullis et al., eds., 1994); “Molecular Evolution”, (Li, 1997).

### DEFINITIONS

It is to be noted that the term “a” or “an” entity refers to one or more of that entity; for example, a gene refers to one or more genes, or at least one gene. As such, the terms “a” (or “an”), “one or more” and “at least one” can be used interchangeably herein. It is also to be noted that the terms “comprising,” “including,” and “having” can be used interchangeably.

As used herein, a “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified polynucleotides such as methylated and/or capped polynucleotides, polynucleotides containing modified bases, backbone modifications, and the like. The terms “polynucleotide” and “nucleotide sequence” are used interchangeably.

As used herein, a “gene” refers to a polynucleotide or portion of a polynucleotide comprising a sequence that encodes a protein. It is well understood in the art that a gene also comprises non-coding sequences, such as 5′ and 3′ flanking sequences (such as promoters, enhancers, repressors, and other regulatory sequences) as well as introns.

The terms “polypeptide,” “peptide,” and “protein” are used interchangeably herein to refer to polymers of amino acids of any length. These terms also include proteins that are post-translationally modified through reactions that include glycosylation, acetylation and phosphorylation.

The term “domesticated organism” refers to an individual living organism or population of same, a species, subspecies, variety, cultivar or strain, that has been subjected to artificial selection pressure and developed a commercially or aesthetically relevant trait. In some preferred embodiments, the domesticated organism is a plant selected from the group consisting of maize, wheat, rice, sorghum, tomato or potato, or any other domesticated plant of commercial interest, where an ancestor or family member is known. A “plant” is any plant at any stage of development, particularly a seed plant.

The term “wild ancestor or family member” or “ancestor or family member” means a forerunner or predecessor organism, species, subspecies, variety, cultivar or strain from which a domesticated organism, species, subspecies, variety, cultivar or strain has evolved. A domesticated organism can have one or more than one ancestor or family member. Typically, domesticated plants can have one or a plurality of ancestor or family members, while domesticated animals usually have only a single ancestor or family member.

The term “commercially or aesthetically relevant trait” is used herein to refer to traits that exist in domesticated organisms such as plants or animals whose analysis could provide information (e.g., physical or biochemical data) relevant to the development of improved organisms or of agents that can modulate the polypeptide responsible for the trait, or the respective polynucleotide. The commercially or aesthetically relevant trait can be unique, enhanced or altered relative to the ancestor or family member. By “altered,” it is meant that the relevant trait differs qualitatively or quantitatively from traits observed in the ancestor or family member. A preferred commercially or aesthetically relevant trait is yield.

The term “K_{A}/K_{S}-type methods” means methods that evaluate differences, frequently (but not always) shown as a ratio, between the number of nonsynonymous substitutions and synonymous substitutions in homologous genes (including the more rigorous methods that determine non-synonymous and synonymous sites). These methods are designated using several systems of nomenclature, including but not limited to K_{A}/K_{S}, d_{N}/d_{S}, D_{N}/D_{S}.

The terms “evolutionarily significant change” and “adaptive evolutionary change” refer to one or more nucleotide or peptide sequence change(s) between two organisms, species, subspecies, varieties, cultivars and/or strains that may be attributed to either relaxation of selective pressure or positive selective pressure. One method for determining the presence of an evolutionarily significant change is to apply a K_{A}/K_{S}-type analytical method, such as to measure a K_{A}/K_{S} ratio. Typically, a K_{A}/K_{S} ratio of 1.0 or greater is considered to be an evolutionarily significant change.

Strictly speaking, K_{A}/K_{S} ratios of exactly 1.0 are indicative of relaxation of selective pressure (neutral evolution), and K_{A}/K_{S} ratios greater than 1.0 are indicative of positive selection. However, it is commonly accepted that the ESTs in GenBank and other public databases often suffer from some degree of sequencing error, and even a few incorrect nucleotides can influence K_{A}/K_{S} ratios. For this reason, polynucleotides with K_{A}/K_{S} ratios as low as 0.75 can be carefully resequenced and re-evaluated for relaxation of selective pressure (neutral evolutionarily significant change), positive selection pressure (positive evolutionarily significant change), or negative selective pressure (evolutionarily conservative change).

The term “positive evolutionarily significant change” means an evolutionarily significant change in a particular organism, species, subspecies, variety, cultivar or strain that results in an adaptive change that is positive as compared to other related organisms. An example of a positive evolutionarily significant change is a change that has resulted in enhanced yield in crop plants. As stated above, positive selection is indicated by a K_{A}/K_{S} ratio greater than 1.0. With increasing preference, the K_{A}/K_{S} value is greater than 1.25, 1.5 and 2.0.

The term “neutral evolutionarily significant change” refers to a polynucleotide or polypeptide change that appears in a domesticated organism relative to its ancestral organism, and which has developed under neutral conditions. A neutral evolutionary change is evidenced by a K_{A}/K_{S} value of between about 0.75-1.25, preferably between about 0.9 and 1.1, and most preferably equal to about 1.0. Also, in the case of neutral evolution, there is no “directionality” to be inferred. The gene is free to accumulate changes without constraint, so both the ancestral and domesticated versions are changing with respect to one another.

The term “homologous” or “homologue” or “ortholog” is known and well understood in the art and refers to related sequences that share a common ancestor or family member and is determined based on degree of sequence identity. These terms describe the relationship between a gene found in one species, subspecies, variety, cultivar or strain and the corresponding or equivalent gene in another species, subspecies, variety, cultivar or strain. For purposes of this invention homologous sequences are compared. “Homologous sequences” or “homologues” or “orthologs” are thought, believed, or known to be functionally related. A functional relationship may be indicated in any one of a number of ways, including, but not limited to, (a) degree of sequence identity; (b) same or similar biological function. Preferably, both (a) and (b) are indicated. The degree of sequence identity may vary, but is preferably at least 50% (when using standard sequence alignment programs known in the art), more preferably at least 60%, more preferably at least about 75%, more preferably at least about 85%. Homology can be determined using software programs readily available in the art, such as those discussed in Current Protocols in Molecular Biology (F. M. Ausubel et al., eds., 1987) Supplement 30, section 7.718, Table 7.71. Preferred alignment programs are MacVector (Oxford Molecular Ltd, Oxford, U.K.) and ALIGN Plus (Scientific and Educational Software, Pennsylvania). Another preferred alignment program is Sequencher (Gene Codes, Ann Arbor, Mich.), using default parameters.

The term “nucleotide change” refers to nucleotide substitution, deletion, and/or insertion, as is well understood in the art.

“Housekeeping genes” is a term well understood in the art and means those genes associated with general cell function, including but not limited to growth, division, stasis, metabolism, and/or death. “Housekeeping” genes generally perform functions found in more than one cell type. In contrast, cell-specific genes generally perform functions in a particular cell type and/or class.

The term “agent”, as used herein, means a biological or chemical compound such as a simple or complex organic or inorganic molecule, a peptide, a protein or an oligonucleotide that modulates the function of a polynucleotide or polypeptide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

The term “to modulate function” of a polynucleotide or a polypeptide means that the function of the polynucleotide or polypeptide is altered when compared to not adding an agent. Modulation may occur on any level that affects function. A polynucleotide or polypeptide function may be direct or indirect, and measured directly or indirectly.

A “function of a polynucleotide” includes, but is not limited to, replication; translation; expression pattern(s). A polynucleotide function also includes functions associated with a polypeptide encoded within the polynucleotide. For example, an agent which acts on a polynucleotide and affects protein expression, conformation, folding (or other physical characteristics), binding to other moieties (such as ligands), activity (or other functional characteristics), regulation and/or other aspects of protein structure or function is considered to have modulated polynucleotide function.

A “function of a polypeptide” includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

The term “target site” means a location in a polypeptide which can be a single amino acid and/or is a part of, a structural and/or functional motif, e.g., a binding site, a dimerization domain, or a catalytic active site. Target sites may be useful for direct or indirect interaction with an agent, such as a therapeutic agent.

The term “molecular difference” includes any structural and/or functional difference. Methods to detect such differences, as well as examples of such differences, are described herein.

A “functional effect” is a term well known in the art, and means any effect which is exhibited on any level of activity, whether direct or indirect.

The term “ease of harvest” refers to plant characteristics or features that facilitate manual or automated collection of structures or portions (e.g., fruit, leaves, roots) for consumption or other commercial processing.

The term “yield” refers to the amount of plant or animal tissue or material that is available for use by humans for food, therapeutic, veterinary or other markets.

The term “enhanced economic productivity” refers to the ability to modulate a commercially or aesthetically relevant trait so as to improve desired features. Increased yield and enhanced stress resistance are two examples of enhanced economic productivity.

### General Procedures Known in the Art

For the purposes of this invention, the source of the polynucleotide from the domesticated plant or its ancestor or family member can be any suitable source, e.g., genomic sequences or cDNA sequences. Preferably, cDNA sequences are compared. Protein-coding sequences can be obtained from available private, public and/or commercial databases such as those described herein. These databases serve as repositories of the molecular sequence data generated by ongoing research efforts. Alternatively, protein-coding sequences may be obtained from, for example, sequencing of cDNA reverse transcribed from mRNA expressed in cells, or after PCR amplification, according to methods well known in the art. Alternatively, genomic sequences may be used for sequence comparison. Genomic sequences can be obtained from available public, private and/or commercial databases or from sequencing of genomic DNA libraries or from genomic DNA, after PCR.

In some embodiments, the cDNA is prepared from mRNA obtained from a tissue at a determined developmental stage, or a tissue obtained after the organism has been subjected to certain environmental conditions. cDNA libraries used for the sequence comparison of the present invention can be constructed using conventional cDNA library construction techniques that are explained fully in the literature of the art. Total mRNAs are used as templates to reverse-transcribe cDNAs. Transcribed cDNAs are subcloned into appropriate vectors to establish a cDNA library. The established cDNA library can be maximized for full-length cDNA contents, although less than full-length cDNAs may be used. Furthermore, the sequence frequency can be normalized according to, for example, Bonaldo et al. (1996) Genome Research 6:791-806. cDNA clones randomly selected from the constructed cDNA library can be sequenced using standard automated sequencing techniques. Preferably, full-length cDNA clones are used for sequencing. Either the entire or a large portion of cDNA clones from a cDNA library may be sequenced, although it is also possible to practice some embodiments of the invention by sequencing as little as a single cDNA, or several cDNA clones.

In one preferred embodiment of the present invention, cDNA clones to be sequenced can be pre-selected according to their expression specificity. In order to select cDNAs corresponding to active genes that are specifically expressed, the cDNAs can be subject to subtraction hybridization using mRNAs obtained from other organs, tissues or cells of the same organism. Under certain hybridization conditions with appropriate stringency and concentration, those cDNAs that hybridize with non-tissue specific mRNAs and thus likely represent “housekeeping” genes will be excluded from the cDNA pool. Accordingly, remaining cDNAs to be sequenced are more likely to be associated with tissue-specific functions. For the purpose of subtraction hybridization, non-tissue-specific mRNAs can be obtained from one tissue, or preferably from a combination of different tissues and cells. The amount of non-tissue-specific mRNAs are maximized to saturate the tissue-specific cDNAs.

Alternatively, information from online databases can be used to select or give priority to cDNAs that are more likely to be associated with specific functions. For example, the ancestral cDNA candidates for sequencing can be selected by PCR using primers designed from candidate domesticated organism cDNA sequences. Candidate domesticated organism cDNA sequences are, for example, those that are only found in a specific portion of a plant, or that correspond to genes likely to be important in the specific function. Such specific cDNA sequences may be obtained by searching online sequence databases in which information with respect to the expression profile and/or biological activity for cDNA sequences may be specified.

Sequences of ancestral homologue(s) to a known domesticated organism's gene may be obtained using methods standard in the art, such as PCR methods (using, for example, GeneAmp PCR System 9700 thermocyclers (Applied Biosystems, Inc.)). For example, ancestral cDNA candidates for sequencing can be selected by PCR using primers designed from candidate domesticated organism cDNA sequences. For PCR, primers may be made from the domesticated organism's sequences using standard methods in the art, including publicly available primer design programs such as PRIMER® (Whitehead Institute). The ancestral sequence amplified may then be sequenced using standard methods and equipment in the art, such as automated sequencers (Applied Biosystems, Inc.). Likewise, ancestor or family members gene mimics can be used to obtain corresponding genes in domesticated organisms.

### Identification of Positively Selected Polynucleotides in Domesticated Organisms

In a preferred embodiment, the methods described herein can be applied to identify the genes that control traits of interest in agriculturally important domesticated plants. Humans have bred domesticated plants for several thousand years without knowledge of the genes that control these traits. Knowledge of the specific genetic mechanisms involved would allow much more rapid and direct intervention at the molecular level to create plants with desirable or enhanced traits.

Humans, through artificial selection, have provided intense selection pressures on crop plants. This pressure is reflected in evolutionarily significant changes between homologous genes of domesticated organisms and their wild ancestor or family members. It has been found that only a few genes, e.g., 10-15 per species, control traits of commercial interest in domesticated crop plants. These few genes have been exceedingly difficult to identify through standard methods of plant molecular biology. The K_{A}/K_{S} and related analyses described herein can identify the genes controlling traits of interest.

For any crop plant of interest, cDNA libraries can be constructed from the domesticated species or subspecies and its wild ancestor or family member. As is described in U.S. Ser. No. 09/240,915, filed Jan. 29, 1999, the cDNA libraries of each are “BLASTed” against each other to identify homologous polynucleotides. Alternatively, the skilled artisan can access commercially and/or publicly available genomic or cDNA databases rather than constructing cDNA libraries.

Next, a K_{A}/K_{S} or related analysis may be conducted to identify selected genes that have rapidly evolved under selective pressure. These genes are then evaluated using standard molecular and transgenic plant methods to determine if they play a role in the traits of commercial or aesthetic interest. Using the methods of the invention, the inventors have identified polynucleotides and polypeptides corresponding to genes EG1117 or EG307, which are yield-related genes. The genes of interest can be manipulated by, e.g., random or site-directed mutagenesis, to develop new, improved varieties, subspecies, strains or cultivars.

Generally, in one embodiment of the present invention, nucleotide sequences are obtained from a domesticated organism and a wild ancestor or family member. The domesticated organism's and ancestor or family member's nucleotide sequences are compared to one another to identify sequences that are homologous. The homologous sequences are analyzed to identify those that have nucleic acid sequence differences between the domesticated organism and ancestor or family member. Then molecular evolution analysis is conducted to evaluate quantitatively and qualitatively the evolutionary significance of the differences. For genes that have been positively selected, outgroup analysis can be done to identify those genes that have been positively selected in the domesticated organism (or in the ancestor or family member). Next, the sequence is characterized in terms of molecular/genetic identity and biological function. Finally, the information can be used to identify agents that can modulate the biological function of the polypeptide encoded by the gene.

The general methods of the invention entail comparing protein-coding nucleotide sequences of ancestral and domesticated organisms. Bioinformatics is applied to the comparison and sequences are selected that contain a nucleotide change or changes that is/are evolutionarily significant change(s). The invention enables the identification of genes that have evolved to confer some evolutionary advantage and the identification of the specific evolved changes. For example, the domesticated organism may be Oryza sativa and the wild ancestor or family member Oryza rufipogon. In the case of the present invention, protein-coding nucleotide sequences were obtained from plant clones by standard sequencing techniques.

Protein-coding sequences of a domesticated organism and its ancestor or family member are compared to identify homologous sequences. Any appropriate mechanism for completing this comparison is contemplated by this invention. Alignment may be performed manually or by software (examples of suitable alignment programs are known in the art). Preferably, protein-coding sequences from an ancestor or family member or family member are compared to the domesticated species sequences via database searches, e.g., BLAST searches. The high scoring “hits,” i.e., sequences that show a significant similarity after BLAST analysis, will be retrieved and analyzed. Sequences showing a significant similarity can be those having at least about 60%, at least about 75%, at least about 80%, at least about 85%, or at least about 90% sequence identity. Preferably, sequences showing greater than about 80% identity are further analyzed. The homologous sequences identified via database searching can be aligned in their entirety using sequence alignment methods and programs that are known and available in the art, such as the commonly used simple alignment program CLUSTAL V by Higgins et al. (1992) CABIOS 8:189-191.

As an example, nucleotide sequences obtained from O. rufipogon can be used as query sequences in a search of O. sativa ESTs in GenBank to identify homologous sequences. It should be noted that a complete protein-coding nucleotide sequence is not required. Indeed, partial cDNA sequences may be compared. Once sequences of interest are identified by the methods described below, further cloning and/or bioinformatics methods can be used to obtain the entire coding sequence for the gene or protein of interest.

Alternatively, the sequencing and homology comparison of protein-coding sequences between the domesticated organism and its ancestor or family member or a family member may be performed simultaneously by using sequencing chip technology. See, for example, Rava et al. U.S. Pat. No. 5,545,531.

The aligned protein-coding sequences of domesticated organism and ancestor or family member or a family member are analyzed to identify nucleotide sequence differences at particular sites. Again, any suitable method for achieving this analysis is contemplated by this invention. If there are no nucleotide sequence differences, the ancestor or family member or family member protein coding sequence is not usually further analyzed. The detected sequence changes are generally, and preferably, initially checked for accuracy. Preferably, the initial checking comprises performing one or more of the following steps, any and all of which are known in the art: (a) finding the points where there are changes between the ancestral and domesticated organism sequences; (b) checking the sequence fluorogram (chromatogram) to determine if the bases that appear unique to the ancestor or family member or domesticated organism correspond to strong, clear signals specific for the called base; (c) checking the domesticated organism hits to see if there is more than one domesticated organism sequence that corresponds to a sequence change. Multiple domesticated organism sequence entries for the same gene that have the same nucleotide at a position where there is a different nucleotide in an ancestor or family member sequence provides independent support that the domesticated sequence is accurate, and that the change is significant. Such changes are examined using database information and the genetic code to determine whether these nucleotide sequence changes result in a change in the amino acid sequence of the encoded protein. As the definition of “nucleotide change” makes clear, the present invention encompasses at least one nucleotide change, either a substitution, a deletion or an insertion, in a protein-coding polynucleotide sequence of a domesticated organism as compared to a corresponding sequence from the ancestor or family member. Preferably, the change is a nucleotide substitution. More preferably, more than one substitution is present in the identified sequence and is subjected to molecular evolution analysis.

In one embodiment, the present invention includes a method for identifying a polynucleotide sequence that is associated with yield in plant. This method includes the step of comparing at least a portion of plant polynucleotide sequence with at least one EG1117 polynucleotide sequence and/or EG307 polynucleotide sequence. This method also includes the step of identifying at least one polynucleotide sequence in the plant that contains at least one nucleotide change as compared to a polynucleotide selected from the group consisting of an EG1117 polynucleotide sequence and an EG307 polynucleotide sequence, wherein said identified polynucleotide sequence is associated with yield in a plant. Preferred EG307 and EG1117 polynucleotide sequences include SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; SEQ ID NO:93; and a polynucleotide having at least about 70% sequence identity to the preceding SEQ ID Nos.

Preferred plant polynucleotide sequence includes plant sequence that is derived from genomic DNA or derived from the expressed genes of a plant, i.e., is cDNA. Methods to do so are known in the art and are discussed elsewhere in the instant specification.

Preferably, the EG307 or EG1117 polynucleotide sequence is associated with increased yield in a plant. Methods to determine and quantitate yields are known in the art, and discussed elsewhere in the present specification. Most preferably, yield may be quantitated by determining whether yield is increased relative to a second plant from a common ancestor, genus, or family member plant, more preferably the same species, even more preferably the same cultivar, having a second EG307 or EG1117 polynucleotide sequence with at least one nucleotide change relative to the EG307 or EG1117 polynucleotide sequence from the plant.

In all embodiments of the present invention, a preferred polynucleotide sequence includes a polynucleotide having at least about 60% sequence identity to a to a EG307 or EG1117 polynucleotide of the present invention and has substantially the same effect on yield as a named SEQ ID NO herein. Preferably, a polynucleotide of the present invention will have at least about 65% identity to, at least about 66% identity to, at least about 67% identity to, at least about 68% identity to, at least about 69% identity to, at least about 70% identity to, at least about 71% identity to, at least about 72% identity to, at least about 73% identity to, at least about 74% identity to, at least about 75% identity to, at least about 76% identity to, at least about 77% identity to, at least about 78% identity to, at least about 79% identity to, at least about 80% identity to, at least about 81% identity to, at least about 82% identity to, at least about 83% identity to, at least about 84% identity to, at least about 85% identity to, at least about 86% identity to, at least about 87% identity to, at least about 88% identity to, at least about 89% identity to, at least about 90% identity to, at least about 91% identity to, more preferably at least about at least about 92% identity to, at least about 93% identity to, at least about 94% identity to, at least about 95% identity to, and even more preferably at least about 95.5% identity to, at least about 96% identity to, at least about 96.5% identity to, at least about 97% identity to, at least about 97.5% identity to, at least about 98% identity to, at least about 98.5% identity to, at least about 99% identity to, at least about 99.5% identity to, or are identical to any of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; and SEQ ID NO:93.

In all embodiments of the present invention, a preferred polypeptide sequence includes a polypeptide having at least about 60% sequence identity to a EG307 or EG1117 polypeptide of the present invention and has substantially the same effect on yield as a named SEQ ID NO herein. Preferably, a polypeptide of the present invention will have at least about 65% identity to, at least about 66% identity to, at least about 67% identity to, at least about 68% identity to, at least about 69% identity to, at least about 70% identity to, at least about 71% identity to, at least about 72% identity to, at least about 73% identity to, at least about 74% identity to, at least about 75% identity to, at least about 76% identity to, at least about 77% identity to, at least about 78% identity to, at least about 79% identity to, at least about 80% identity to, at least about 81% identity to, at least about 82% identity to, at least about 83% identity to, at least about 84% identity to, at least about 85% identity to, at least about 86% identity to, at least about 87% identity to, at least about 88% identity to, at least about 89% identity to, at least about 90% identity to, at least about 91% identity to, more preferably at least about at least about 92% identity to, at least about 93% identity to, at least about 94% identity to, at least about 95% identity to, and even more preferably at least about 95.5% identity to, at least about 96% identity to, at least about 96.5% identity to, at least about 97% identity to, at least about 97.5% identity to, at least about 98% identity to, at least about 98.5% identity to, at least about 99% identity to, at least about 99.5% identity to, or are identical to any of SEQ ID NO:73; SEQ ID NO:76; SEQ ID NO:43; SEQ ID NO:46; SEQ ID NO:49; SEQ ID NO:52; SEQ ID NO:55; SEQ ID NO:58; SEQ ID NO:61; SEQ ID NO:64; SEQ ID NO:67; SEQ ID NO:70; SEQ ID NO:118; SEQ ID NO:120; SEQ ID NO:116; SEQ ID NO:96; SEQ ID NO:99; SEQ ID NO:102; SEQ ID NO:104; SEQ ID NO:106; SEQ ID NO:108; SEQ ID NO:110; SEQ ID NO:112; and SEQ ID NO:114.

In all embodiments of the present invention, the domesticated plants of the present invention preferably include Zea mays mays, Oryza sativa, Triticum aestivum, Hordeum vulgare, Saccharum officinarum, Sorghum bicolor, and Pennisetum typhoides. In all embodiments of the present invention, the wild ancestor or family member plants preferably include wild ancestor or family member plants for a domesticated plant selected from the group consisting of Zea mays mays, Oryza sativa, Triticum aestivum, Hordeum vulgare, Saccharum officinarum, Sorghum bicolor, and Pennisetum typhoides. A particularly preferred wild ancestor or family member plant is Oryza rufipogon. Any plant EG307 or EG1117 polypeptide is a suitable polypeptide of the present invention. Suitable plants from which to isolate EG307 or EG1117 polypeptides (including isolation of the natural polypeptide or production of the polypeptide by recombinant or synthetic techniques) include maize, wheat, barley, rye, millet, chickpea, lentil, flax, olive, fig almond, pistachio, walnut, beet, parsnip, citrus fruits, including, but not limited to, orange, lemon, lime, grapefruit, tangerine, minneola, and tangelo, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis, and woody plants such as coniferous and deciduous trees, with corn, sorghum, sugarcane, and wheat being especially desirable.

This embodiment of the present invention includes methods for identifying allelic variants of the sequences of the present invention. As used herein, “marker” includes reference to a locus on a chromosome that serves to identify a unique position on the chromosome. A “polymorphic marker” includes reference to a marker which appears in multiple forms (alleles) such that different forms of the marker, when they are present in a homologous pair, allow transmission of each of the chromosomes in that pair to be followed. A genotype may be defined by use of one or a plurality of markers.

The present invention also provides isolated nucleic acids comprising polynucleotides of sufficient length and complementarity to a gene of the present invention to use as probes or amplification primers in the detection, quantitation, or isolation of gene transcripts. For example, isolated nucleic acids of the present invention can be used as probes in detecting deficiencies in the level of mRNA in screenings for desired transgenic plants, for detecting mutations in the gene (e.g., substitutions, deletions, or additions), for monitoring upregulation of expression or changes in enzyme activity in screening assays of compounds, for detection of any number of allelic variants (polymorphisms) of the gene, or for use as molecular markers in plant breeding programs.

Additionally, the present invention further provides isolated nucleic acids comprising polynucleotides encoding one or more polymorphic (allelic) variants of polypeptides/polynucleotides. Polymorphic variants are frequently used to follow segregation of chromosomal regions in, for example, marker assisted selection methods for crop improvement.

The present invention provides a method of genotyping a plant utilizing polynucleotides of the present invention. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. See, e.g., PELEMAN AND VAN DER VOORT, (2003) TRENDS IN PLANT SCIENCE VOL 8(7):330-334 AND HOLLAND (2004) PROCEEDINGS OF THE 4^{TH} INTERNATIONAL CROP SCIENCE CONGRESS 26 SEP.-1 OCT. 2004, BRISBANE, AUSTRALIA.

The particular method of genotyping in the present invention may employ any number of molecular marker analytic techniques such as, but not limited to, restriction fragment length polymorphisms (RFLPs). RFLPs are the product of allelic differences between DNA restriction fragments caused by nucleotide. sequence variability. As is well known to those of skill in the art, RFLPs are typically detected by extraction of genomic DNA and digestion with a restriction enzyme. Generally, the resulting fragments are separated according to size and hybridized with a probe; single copy probes are suitable. Restriction fragments from homologous chromosomes are revealed. Differences in fragment size among alleles represent an RFLP. Thus, the present invention further provides a means to follow segregation of a gene or nucleic acid of the present invention as well as chromosomal sequences genetically linked to these genes or nucleic acids using such techniques as RFLP analysis. Linked chromosomal sequences are within 50 centiMorgans (cM), often within 40 or 30 cM, in some cases within 20 or 10 cM, and in some cases within 5, 3, 2, or 1 cM of a gene of the present invention.

In the present invention, the nucleic acid probes employed for molecular marker mapping of plant nuclear genomes selectively hybridize, under selective hybridization conditions, to a gene encoding a polynucleotide of the present invention. In some embodiments, the probes are selected from polynucleotides of the present invention. Typically, these probes are cDNA probes or Pst I genomic clones. The length of the probes is discussed in greater detail, supra, but are typically at least 15 bases in length, and in some cases at least 20, 25, 30, 35, 40, or 50 bases in length. Generally, however, the probes are less than about 1 kilobase in length. In some embodiments, the probes are single copy probes that hybridize to a unique locus in a haploid chromosome complement. Some exemplary restriction enzymes employed in RFLP mapping are EcoRI, EcoRV, and Sstl. As used herein the term “restriction enzyme” includes reference to a composition that recognizes and, alone or in conjunction with another composition, cleaves at a specific nucleotide sequence.

The method of detecting an RFLP comprises the steps of (a) digesting genomic DNA of a plant with a restriction enzyme; (b) hybridizing a nucleic acid probe, under selective hybridization conditions, to a sequence of a polynucleotide of the present of said genomic DNA; (c) detecting therefrom a RFLP. Other methods of differentiating polymorphic (allelic) variants of polynucleotides of the present invention can be had by utilizing molecular marker techniques well known to those of skill in the art including such techniques as: 1) single stranded conformation analysis (SSCP); 2) denaturing gradient gel electrophoresis (DGGE); 3) RNase protection assays; 4) allele-specific oligonucleotides (ASOs); 5) the use of proteins which recognize nucleotide mismatches, such as the E. coli mutS protein; and 6) allele-specific PCR. Other approaches based on the detection of mismatches between the two complementary DNA strands include clamped denaturing gel electrophoresis (CDGE); heteroduplex analysis (HA); and chemical mismatch cleavage (CMC). Exemplary polymorphic variants are provided in Table I, below:

TABLE I
Polymorphic variants of EG307 in corn

| High-yield corn hybrids | SEQ ID NOs: 71, 72, 74, 75 |
|---|---|
| Low-yield corn landraces | SEQ ID NOs: 41, 42, 44, 45, |
| and teosinte | 47, 48, 50, 51, 53, 54, 56, 57, |
| | 59, 60, 62, 63, 65, 66, 68, 69 |

Thus, the present invention further provides a method of genotyping comprising the steps of contacting, under stringent hybridization conditions, a sample suspected of comprising a polynucleotide of the present invention with a nucleic acid probe. Generally, the sample is a plant sample; a sample suspected of comprising a maize polynucleotide of the present invention (e.g., gene, mRNA). The nucleic acid probe selectively hybridizes, under stringent conditions, to a subsequence of a polynucleotide of the present invention comprising a polymorphic marker. Selective hybridization of the nucleic acid probe to the polymorphic marker nucleic acid sequence yields a hybridization complex. Detection of the hybridization complex indicates the presence of that polymorphic marker in the sample. In some embodiments, the nucleic acid probe comprises a polynucleotide of the present invention.

It is apparent to those skilled in the art that polymorphic variants can be identified for EG307 and EG 1117 by sequencing these genes.

It is clear to one skilled in the art that additional polymorphic variants or alleles of EG307 and EG1117 can be identified by sequencing more corn lines and hybrids, more rice lines and hybrids, more sorghum, barley, wheat lines, millet, or sugar cane lines and association tests can be performed to find the alleles of each of these two genes that are associated with the best phenotype for yield traits (such as total yield, grain weight, grain length, or other yield related traits) or quality traits (such as ASV, chalk, or other quality traits). Association tests with these additional alleles would indicate which alleles are associated with desired phenotypes for specific traits. Prospective parent inbred lines could then be screened for either the presence of the alleles (or portions of the desired alleles that are diagnostic) associated with best performance for a yield trait (such as total yield, grain weight, grain length, grains per plant, etc.) or best performance for a quality trait (such as ASV or chalk, etc.). Alleles associated with the best performance for a yield trait or a quality trait would be the “desired allele” for attaining the desired phenotype.

In preferred embodiments, the present invention provides methods for identifying alleles of EG307 or EG 1117 in a crop species; methods for determining whether a plant contains a preferred allele of EG307 or EG1117, and methods for screening plants for preferred alleles of EG307 or EG1117. Alleles of EG307 and EG1117 include, for example, SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; SEQ ID NO:93; and a polynucleotide having at least about 70% sequence identity to a polynucleotide enumerated above.

For methods to identify other alleles of EG307 or EG1117, methods include in one step, using at least a portion of any sequence from the polynucleotide sequences of the present invention to amplify the corresponding EG307 or EG1117 sequence in one or more plants of a crop species. In another step, these methods include determining the nucleotide sequence of amplified sequences. In another step, these methods include comparing the amplified sequences to polynucleotide sequences of the present invention to identify any alleles of EG307 or EG1117 in the tested plants of the crop species.

Generally, these methods also include methods for identifying or determining preferred alleles (e.g., alleles that are associated with a desired trait). In one step, using at least a portion of any sequence from the polynucleotide sequences of the present invention to amplify the corresponding EG307 or EG1117 sequence in at least two plants for which a particular parameter for a trait has been or can be measured. Such a trait includes yield, for example. In another step, these methods include determining the sequence of EG307 or EG1117 in each plant. In another step, these methods include identifying preferred alleles or polynucleotide sequences of EG307 or EG1117. Preferred alleles may be identified by genotyping analysis by determining the association of the allele with the desired trait. Examples of such genotyping analysis can be found herein in the Examples.

Generally, these methods also include methods for screening plants for preferred alleles or polynucleotide sequences. Such methods include using at least a portion of a preferred allele (e.g., alleles associated with a desired trait) to amplify the corresponding EG307 or EG1117 sequence in a plant, and select those plants that contain the desired allele (or polynucleotide sequence). The present invention also provides a method of producing an EG307 or EG1117 polypeptide comprising: a) providing a cell transfected with a polynucleotide encoding an EG307 or EG1117 polypeptide positioned for expression in the cell; b) culturing the transfected cell under conditions for expressing the polynucleotide; and c) isolating the EG307 or EG1117 polypeptide.

The present invention also provides a method of isolating a yield-related gene from a recombinant plant cell library. The method includes providing a preparation of plant cell DNA or a recombinant plant cell library; contacting the preparation or plant cell library with a detectably-labeled EG307 or EG1117 conserved oligonucleotides (generated from an EG307 or EG1117 polynucleotide sequence of the present invention, as described elsewhere herein) under hybridization conditions providing detection of genes having 50% or greater sequence identity; and isolating a yield-related gene by its association with the detectable label.

The present invention also provides a method of isolating a yield-related gene from plant cell DNA. The method includes providing a sample of plant cell DNA; providing a pair of oligonucleotides having sequence homology to a conserved region of an EG307 or EG1117 gene oligonucleotides (generated from an EG307 or EG1117 polynucleotide sequence of the present invention, as described elsewhere herein); combining the pair of oligonucleotides with the plant cell DNA sample under conditions suitable for polymerase chain reaction-mediated DNA amplification; and isolating the amplified yield-related gene or fragment thereof.

The sequences identified by the methods described herein can be used to identify agents that are useful in modulating domesticated organism-unique, enhanced or altered functional capabilities and/or correcting defects in these capabilities using these sequences. These methods employ, for example, screening techniques known in the art, such as in vitro systems, cell-based expression systems and transgenic animals and plants. The approach provided by the present invention not only identifies rapidly evolved genes, but indicates modulations that can be made to the protein that may not be too toxic because they exist in another species.

The present invention also provides a method of producing an EG307 or EG1117 polypeptide. Steps include providing a cell transfected with a polynucleotide encoding an EG307 or EG1117 polypeptide positioned for expression in the cell; and culturing the transfected cell under conditions for expressing the polynucleotide; and c) isolating the EG307 or EG1117 polypeptide.

The present invention also provides a method of detecting a yield-increasing gene or a yield-increasing allelic variant of a gene in a plant cell which includes the following steps. Steps include contacting the EG307 or EG1117 gene or a portion thereof greater than 12 nucleotides, in some cases greater than 30 nucleotides in length with a preparation of genomic DNA from the plant cell under hybridization conditions providing detection of nucleic acid molecule sequences having about 50% or greater sequence identity to a EG307 or EG1117 polynucleotide of the present invention, such as, for example, a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; SEQ ID NO:93; and a polynucleotide having at least about 70% sequence identity to a polynucleotide enumerated above; and detecting hybridization, whereby a yield-increasing gene may be identified.

The present invention also provides a method of detecting a yield-increasing gene or a specific yield increasing allelic variant of a gene in a plant cell. This method includes contacting the yield increasing genes EG307 or EG1117 or a portion of any of these genes greater than 12 nucleotides, in some cases greater than 30 nucleotides in length with a preparation of genomic DNA from the plant cell under hybridization conditions providing detection of nucleic acid molecule sequences having about 50% or greater sequence identity to the polynucleotides of the present invention as described elsewhere herein; and detecting hybridization, whereby a yield-increasing gene or a specific yield increasing allelic variant of a gene may be identified.

The sequences identified by the methods described herein can be used to identify agents that are useful in modulating domesticated organism-unique, enhanced or altered functional capabilities and/or correcting defects in these capabilities using these sequences. These methods employ, for example, screening techniques known in the art, such as in vitro systems, cell-based expression systems and transgenic animals and plants. The approach provided by the present invention not only identifies rapidly evolved genes, but indicates modulations that can be made to the protein that may not be too toxic because they exist in another species.

In one embodiment, the present invention includes a method of determining whether a plant has a particular polynucleotide sequence comprising an EG307 sequence. This method includes the following steps. One step includes comparing at least about a portion of polypeptide-coding nucleotide sequence of said plant with a polynucleotide sequence of an EG307 polynucleotide of the present invention, such as, for example, those selected from the group consisting of (i) a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; and SEQ ID NO:85; and (ii) a polynucleotide having at least about 70% sequence identity to a polynucleotide of (i) and which confers substantially the same yield as a polynucleotide of (i). One of the polynucleotides enumerated above can be selected as the particular polynucleotide (i.e., the polynucleotide of interest, for the determination of whether the plant contains that polynucleotide or a related one.) In another step, the method includes identifying whether the plant contains the particular polynucleotide. Preferably, the plant polynucleotide sequence is genomic DNA or cDNA.

In another embodiment, the present invention includes a method of determining whether a plant has a particular polynucleotide sequence comprising an EG1117 sequence. This method includes the step of comparing at least about a portion of the polynucleotide sequence of said plant with a EG 1117 polynucleotide sequence of the present invention, such as, for example, a polynucleotide selected from the group consisting of (i) a polynucleotide selected from the group consisting of SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; and SEQ ID NO:93 and (ii) a polynucleotide having at least about 70% sequence identity to a polynucleotide of (i) and which confers substantially the same yield as a polynucleotide of (i). One of the polynucleotides enumerated above can be selected as the particular polynucleotide (i.e., the polynucleotide of interest, for the determination of whether the plant contains that polynucleotide or a related one.) In another step, the method includes identifying whether the plant contains the particular polynucleotide.

Preferably, the plant polynucleotide sequence is genomic DNA or cDNA. Preferably, the EG307 or EG1117 polynucleotide sequence is associated with increased yield in a plant. Methods to determine and quantitate yields are known in the art, and discussed elsewhere in the present specification. For example, increased yield may be increased yield relative to a second plant from a common ancestor, genus or family member plant having a second EG307 polynucleotide sequence with at least one nucleotide change relative to the EG307 polynucleotide sequence from the plant.

The present invention also provides methods of modifying the frequency of a grain yield gene in a plant population, and methods for marker assisted breeding or marker assisted selection which includes the following steps. One step includes screening a plurality of plants using an oligonucleotide as a marker to determine the presence or absence of a grain filling gene in an individual plant, the oligonucleotide consisting of not more than 300 bases of a nucleotide sequence selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; SEQ ID NO:93, and a polynucleotide having at least about 70% sequence identity to a preceding SEQ ID No. Another step includes selecting at least one individual plant for breeding based on the presence or absence of the grain yield gene; and another step includes breeding at least one plant thus selected to produce a population of plants having a modified frequency of the grain yield gene.

In one embodiment, methods for marker assisted breeding include a method of marker assisted breeding of plants for a particular EG1117 polynucleotide sequence. This embodiment includes the following steps. One step includes comparing, for at least one plant, at least a portion of the nucleotide sequence of said plants with the particular EG1117 polynucleotide sequence of the present invention, such as, for example, those selected from the group consisting of (i) a polynucleotide selected from the group consisting of SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; and SEQ ID NO:93; and (ii) a polynucleotide having at least about 70% sequence identity to a polynucleotide of (i) and which confers substantially the same yield as a polypeptide of (i). This method also includes the step of identifying whether the plant comprises the particular polynucleotide sequence; and the step of breeding a plant comprising the particular polynucleotide sequence to produce progeny.

Methods for marker assisted breeding also include a method of marker assisted breeding of plants for a particular EG307 polynucleotide sequence. Steps include comparing, for at least one plant, at least a portion of the nucleotide sequence of said plants with a particular EG307 of the present invention, such as, for example, a polynucleotide sequence selected from the group consisting of (i) a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; and SEQ ID NO:85; and (ii) a polynucleotide having at least about 70% sequence identity to a polynucleotide of (i) and which confers substantially the same yield as a polypeptide of (i), identifying whether the plant comprises the particular polynucleotide sequence; and breeding a plant comprising the particular polynucleotide sequence to produce progeny.

These marker assisted breeding methods include a method for selecting plants, for example cereals (including, but not limited to maize, wheat, barley and other members of the Grass family) or legumes (for example, soy beans), having an altered yield comprising obtaining nucleic acid molecules from the plants to be selected, contacting the nucleic acid molecules with one or more probes that selectively hybridize under stringent or highly stringent conditions to a nucleic acid sequence comprising the EG307 and EG1117 polynucleotides of the present invention; detecting the hybridization of the one or more probes to the nucleic acid sequences wherein the presence of the hybridization indicates the presence of a gene associated with altered yield; and selecting plants on the basis of the presence or absence of such hybridization. In one embodiment, marker-assisted selection is accomplished in rice. In another embodiment, marker assisted selection is accomplished in wheat using one or more probes which selectively hybridize under stringent or highly stringent conditions to sequences comprising the EG307 and EG1117 polynucleotides of the present invention. In yet another embodiment, marker assisted selection is accomplished in maize or corn using one or more probes which selectively hybridize under stringent or highly stringent conditions to polynucleotides comprising the EG307 and EG1117 polynucleotides of the present invention. In still another embodiment, marker assisted selection is accomplished in sorghum using one or more probes which selectively hybridize under stringent or highly stringent conditions to sequences comprising the EG307 and EG1117 polynucleotides of the present invention. In still another embodiment, marker assisted selection is accomplished in barley using one or more probes which selectively hybridize under stringent or highly stringent conditions to sequences comprising the EG307 and EG1117 polynucleotides of the present invention. In each case marker-assisted selection can be accomplished using a probe or probes to a single sequence or multiple sequences. If multiple sequences are used they can be used simultaneously or sequentially.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the genome of the recurrent parent and against the markers of the donor parent. Using this procedure can minimize the amount of genome from the donor parent that remains in the selected plants. It can also be used to reduce the number of crosses back to the recurrent parent needed in a backcrossing program. The use of molecular markers in the selection process is often called. Genetic Marker Enhanced Selection.

In another embodiment, the present invention includes an isolated polynucleotide which includes one or more of the following polynucleotides: SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; SEQ ID NO:93; and a polynucleotide sequence having at least about 70% sequence identity to a (i.e., any) polynucleotide sequence enumerated above and confers substantially the same yield as any polynucleotide sequence enumerated above.

One embodiment of the present invention is an isolated plant polynucleotide that hybridizes under stringent hybridization conditions with at least one of the following genes: an EG307 or EG1117gene. The identifying characteristics of such genes are heretofore described. A polynucleotide of the present invention can include an isolated natural plant EG307 or EG1117gene or a homologue thereof, the latter of which is described in more detail below. A polynucleotide of the present invention can include one or more regulatory regions, full-length or partial coding regions, or combinations thereof. The minimal size of a polynucleotide of the present invention is the minimal size that can form a stable hybrid with one of the aforementioned genes under stringent hybridization conditions. Suitable plants are disclosed above.

In accordance with the present invention, an isolated polynucleotide is a polynucleotide that has been removed from its natural milieu (i.e., that has been subject to human manipulation). As such, “isolated” does not reflect the extent to which the polynucleotide has been purified. An isolated polynucleotide can include DNA, RNA, or derivatives of either DNA or RNA.

An isolated plant EG307 or EG1117 polynucleotide of the present invention can be obtained from its natural source either as an entire (i.e., complete) gene or a portion thereof capable of forming a stable hybrid with that gene. An isolated plant EG307 or EG1117 polynucleotide can also be produced using recombinant DNA technology (e.g., polymerase chain reaction (PCR) amplification, cloning) or chemical synthesis. Isolated plant EG307 or EG1117 polynucleotides include natural polynucleotides and homologues thereof, including, but not limited to, natural allelic variants and modified polynucleotides in which nucleotides have been inserted, deleted, substituted, and/or inverted in such a manner that such modifications do not substantially interfere with the polynucleotide's ability to encode an EG307 or EG1117 polypeptide of the present invention or to form stable hybrids under stringent conditions with natural gene isolates.

Once the desired DNA has been isolated, it can be sequenced by known methods. It is recognized in the art that such methods are subject to errors, such that multiple sequencing of the same region is routine and is still expected to lead to measurable rates of mistakes in the resulting deduced sequence, particularly in regions having repeated domains, extensive secondary structure, or unusual base compositions, such as regions with high GC base content. When discrepancies arise, resequencing can be done and can employ special methods. Special methods can include altering sequencing conditions by using: different temperatures; different enzymes; proteins which alter the ability of oligonucleotides to form higher order structures; altered nucleotides such as ITP or methylated dGTP; different gel compositions, for example adding formamide; different primers or primers located at different distances from the problem region; or different templates such as single stranded DNAs. Sequencing of mRNA can also be employed. The inventors note that SEQ ID NO: 97, an EG1117 polynucleotide from S. bicolor, originally disclosed in U.S. Ser. No. 60/666,511 as SEQ ID NO:3, was found to have an error and has been corrected, so that SEQ ID NO:97 is the correct version to the best of the inventor's current knowledge.

A plant EG307 or EG1117 polynucleotide homologue can be produced using a number of methods known to those skilled in the art (see, for example, Sambrook et al., ibid.). For example, polynucleotides can be modified using a variety of techniques including, but not limited to, classic mutagenesis techniques and recombinant DNA techniques, such as site-directed mutagenesis, chemical treatment of a polynucleotide to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of polynucleotides and combinations thereof. Polynucleotide homologues can be selected from a mixture of modified nucleic acids by screening for the function of the polypeptide encoded by the nucleic acid (e.g., ability to elicit an immune response against at least one epitope of an EG307 or EG1117 polypeptide, ability to increase yield in a transgenic plant containing an EG307 or EG1117gene) and/or by hybridization with an EG307 or EG1117gene.

An isolated polynucleotide of the present invention can include a nucleic acid sequence that encodes at least one plant EG307 or EG1117 polypeptide of the present invention, examples of such polypeptides being disclosed herein. Although the phrase “polynucleotide” primarily refers to the physical polynucleotide and the phrase “nucleic acid sequence” primarily refers to the sequence of nucleotides on the polynucleotide, the two phrases can be used interchangeably, especially with respect to a polynucleotide, or a nucleic acid sequence, being capable of encoding an EG307 or EG1117 polypeptide. As heretofore disclosed, plant EG307 or EG1117 polypeptides of the present invention include, but are not limited to, polypeptides having full-length plant EG307 or EG1117coding regions, polypeptides having partial plant EG307 or EG1117coding regions, fusion polypeptides, multivalent protective polypeptides and combinations thereof.

At least certain polynucleotides of the present invention encode polypeptides that selectively bind to immune serum derived from an animal that has been immunized with an EG307 or EG1117 polypeptide from which the polynucleotide was isolated.

A polynucleotide of the present invention, when expressed in a suitable plant, is capable of increasing the yield of the plant. As will be disclosed in more detail below, such a polynucleotide can be, or encode, an antisense RNA, a molecule capable of triple helix formation, a ribozyme, or other nucleic acid-based compound.

One embodiment of the present invention is a plant EG307 or EG1117 polynucleotide that hybridizes under stringent hybridization conditions to an EG307 or EG1117 polynucleotide of the present invention, or to a homologue of such an EG307 or EG1117 polynucleotide, or to the complement of such a polynucleotide. A polynucleotide complement of any nucleic acid sequence of the present invention refers to the nucleic acid sequence of the polynucleotide that is complementary to (i.e., can form a complete double helix with) the strand for which the sequence is cited. It is to be noted that a double-stranded nucleic acid molecule of the present invention for which a nucleic acid sequence has been determined for one strand, that is represented by a SEQ ID NO, also comprises a complementary strand having a sequence that is a complement of that SEQ ID NO. As such, polynucleotides of the present invention, which can be either double-stranded or single-stranded, include those polynucleotides that form stable hybrids under stringent hybridization conditions with either a given SEQ ID NO denoted herein and/or with the complement of that SEQ ID NO, which may or may not be denoted herein. Methods to deduce a complementary sequence are known to those skilled in the art. In some embodiments an EG307 or EG1117 polynucleotide is capable of encoding at least a portion of an EG307 or EG1117 polypeptide that naturally is present in plants.

In some embodiments, EG307 or EG1117 polynucleotides of the present invention hybridize under stringent hybridization conditions with at least one of the following polynucleotides: SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; SEQ ID NO:93, or to a homologue or complement of such polynucleotide.

Knowing the nucleic acid sequences of certain plant EG307 or EG1117 polynucleotides of the present invention allows one skilled in the art to, for example, (a) make copies of those polynucleotides, (b) obtain polynucleotides including at least a portion of such polynucleotides (e.g., polynucleotides including full-length genes, full-length coding regions, regulatory control sequences, truncated coding regions), and (c) obtain EG307 or EG1117 polynucleotides for other plants. Such polynucleotides can be obtained in a variety of ways including screening appropriate expression libraries with antibodies of the present invention; traditional cloning techniques using oligonucleotide probes of the present invention to screen appropriate libraries or DNA; and PCR amplification of appropriate libraries or DNA using oligonucleotide primers of the present invention. Suitable libraries to screen or from which to amplify polynucleotides include libraries such as genomic DNA libraries, BAC libraries, YAC libraries, cDNA libraries prepared from isolated plant tissues, including, but not limited to, stems, reproductive structures/tissues, leaves, roots, and tillers; and libraries constructed from pooled cDNAs from any or all of the tissues listed above. In the case of rice and corn, BAC libraries, available from Clemson University may be used. Similarly, DNA sources to screen or from which to amplify polynucleotides include plant genomic DNA. Techniques to clone and amplify genes are disclosed, for example, in Sambrook et al., ibid. and in Galun & Breiman, TRANSGENIC PLANTS, Imperial College Press, 1997.

The present invention also includes polynucleotides that are oligonucleotides capable of hybridizing, under stringent hybridization conditions, with complementary regions of other, sometimes longer, polynucleotides of the present invention such as those comprising plant EG307 or EG1117genes or other plant EG307 or EG1117 polynucleotides. Oligonucleotides of the present invention can be RNA, DNA, or derivatives of either. The minimal size of such oligonucleotides is the size required to form a stable hybrid between a given oligonucleotide and the complementary sequence on another polynucleotide of the present invention. Minimal size characteristics are disclosed herein. The size of the oligonucleotide must also be sufficient for the use of the oligonucleotide in accordance with the present invention. Oligonucleotides of the present invention can be used in a variety of applications including, but not limited to, as probes to identify additional polynucleotides, as primers to amplify or extend polynucleotides, as targets for expression analysis, as candidates for targeted mutagenesis and/or recovery, or in agricultural applications to alter EG307 or EG1117 polypeptide production or activity. Such agricultural applications include the use of such oligonucleotides in, for example, antisense-, triplex formation-, ribozyme- and/or RNA drug-based technologies. The present invention, therefore, includes such oligonucleotides and methods to enhance economic productivity in a plant by use of one or more of such technologies.

The present invention also includes an isolated polypeptide which includes one or more of a polypeptide encoded by the polynucleotides SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113 and a polypeptide encoded by a polynucleotide having at least about 70% sequence identity to a polynucleotide enumerated above and confers substantially the same yield as a polynucleotide enumerated above. Isolated polypeptides of the present invention also include SEQ ID NO:73; SEQ ID NO:76; SEQ ID NO:43; SEQ ID NO:46; SEQ ID NO:49; SEQ ID NO:52; SEQ ID NO:55; SEQ ID NO:58; SEQ ID NO:61; SEQ ID NO:64; SEQ ID NO:67; SEQ ID NO:70; SEQ ID NO:118; SEQ ID NO:120; SEQ ID NO:116; SEQ ID NO:96; SEQ ID NO:99; SEQ ID NO:102; SEQ ID NO:104; SEQ ID NO:106; SEQ ID NO:108; SEQ ID NO:110; SEQ ID NO:112; SEQ ID NO:114; and a polypeptide having at least about 75% sequence identity to any polypeptide enumerated above and confers substantially the same yield as any of the polypeptides enumerated above.

According to the present invention, an isolated, or biologically pure, polypeptide, is a polypeptide that has been removed from its natural milieu. As such, “isolated” and “biologically pure” do not necessarily reflect the extent to which the polypeptide has been purified. An isolated EG307 or EG1117 polypeptide of the present invention can be obtained from its natural source, can be produced using recombinant DNA technology or can be produced by chemical synthesis. An EG307 or EG1117 polypeptide of the present invention may be identified by its ability to perform the function of natural EG307 or EG1117 in a functional assay. By “natural EG307 or EG1117 polypeptide,” it is meant the full length EG307 or EG1117 polypeptide. The phrase “capable of performing the function of a natural EG307 or EG1117 in a functional assay” means that the polypeptide has at least about 10% of the activity of the natural polypeptide in the functional assay. In other embodiments, the EG307 or EG1117 polypeptide has at least about 20% of the activity of the natural polypeptide in the functional assay. In other embodiments, the EG307 or EG1117 polypeptide has at least about 30% of the activity of the natural polypeptide in the functional assay. In other embodiments, the EG307 or EG1117 polypeptide has at least about 40% of the activity of the natural polypeptide in the functional assay. In other embodiments, the EG307 or EG1117 polypeptide has at least about 50% of the activity of the natural polypeptide in the functional assay. In other embodiments, the polypeptide has at least about 60% of the activity of the natural polypeptide in the functional assay. In other embodiments, the polypeptide has at least about 70% of the activity of the natural polypeptide in the functional assay. In other embodiments, the polypeptide has at least about 80% of the activity of the natural polypeptide in the functional assay. In other embodiments, the polypeptide has at least about 90% of the activity of the natural polypeptide in the functional assay. Examples of functional assays include antibody-binding assays, or yield-increasing assays, as detailed elsewhere in this specification.

As used herein, an isolated plant EG307 or EG1117 polypeptide can be a full-length polypeptide or any homologue of such a polypeptide. Examples of EG307 or EG1117 homologues include EG307 or EG1117 polypeptides in which amino acids have been deleted (e.g., a truncated version of the polypeptide, such as a peptide), inserted, inverted, substituted and/or derivatized (e.g., by glycosylation, phosphorylation, acetylation, myristylation, prenylation, palmitoylation, amidation and/or addition of glycerophosphatidyl inositol) such that the homolog has natural EG307 or EG1117 activity.

In one embodiment, when the homologue is administered to an animal as an immunogen, using techniques known to those skilled in the art, the animal will produce a humoral and/or cellular immune response against at least one epitope of a EG307 or EG1117 polypeptide. EG307 or EG1117 homologues can also be selected by their ability to perform the function of EG307 or EG1117 in a functional assay.

Plant EG307 or EG1117 polypeptide homologues can be the result of natural allelic variation or natural mutation. EG307 or EG1117 polypeptide homologues of the present invention can also be produced using techniques known in the art including, but not limited to, direct modifications to the polypeptide or modifications to the gene encoding the polypeptide using, for example, classic or recombinant DNA techniques to effect random or targeted mutagenesis.

In accordance with the present invention, a mimetope refers to any compound that is able to mimic the ability of an isolated plant EG307 or EG1117 polypeptide of the present invention to perform the function of EG307 or EG1117 polypeptide of the present invention in a functional assay. Examples of mimetopes include, but are not limited to, anti-idiotypic antibodies or fragments thereof; that include at least one binding site that mimics one or more epitopes of an isolated polypeptide of the present invention; non-polypeptideaceous immunogenic portions of an isolated polypeptide (e.g., carbohydrate structures); and synthetic or natural organic molecules, including nucleic acids, that have a structure similar to at least one epitope of an isolated polypeptide of the present invention. Such mimetopes can be designed using computer-generated structures of polypeptides of the present invention. Mimetopes can also be obtained by generating random samples of molecules, such as oligonucleotides, peptides or other organic molecules, and screening such samples by affinity chromatography techniques using the corresponding binding partner.

The minimal size of an EG307 or EG1117 polypeptide homologue of the present invention is a size sufficient to be encoded by a polynucleotide capable of forming a stable hybrid with the complementary sequence of a polynucleotide encoding the corresponding natural polypeptide. As such, the size of the polynucleotide encoding such a polypeptide homologue is dependent on nucleic acid composition and percent homology between the polynucleotide and complementary sequence as well as upon hybridization conditions per se (e.g., temperature, salt concentration, and formamide concentration). It should also be noted that the extent of homology required to form a stable hybrid can vary depending on whether the homologous sequences are interspersed throughout the polynucleotides or are clustered (i.e., localized) in distinct regions on the polynucleotides. The minimal size of such polynucleotides is typically at least about 12 to about 15 nucleotides in length if the polynucleotides are GC-rich and at least about 15 to about 17 bases in length if they are AT-rich. In some embodiments, the polynucleotide is at least 12 bases in length. A plant EG307 or EG1117 polypeptide of the present invention is a compound that when expressed or modulated in a plant, is capable of increasing the yield of the plant.

One embodiment of the present invention is a fusion polypeptide that includes EG307 or EG1117 polypeptide-containing domain attached to a fusion segment. Inclusion of a fusion segment as part of an EG307 or EG1117 polypeptide of the present invention can enhance the polypeptide's stability during production, storage and/or use. Depending on the segment's characteristics, a fusion segment can also act as an immunopotentiator to enhance the immune response mounted by an animal immunized with an EG307 or EG1117 polypeptide containing such a fusion segment. Furthermore, a fusion segment can function as a tool to simplify purification of an EG307 or EG1117 polypeptide, such as to enable purification of the resultant fusion polypeptide using affinity chromatography. A suitable fusion segment can be a domain of any size that has the desired function (e.g., imparts increased stability, imparts increased immunogenicity to a polypeptide, and/or simplifies purification of a polypeptide). It is within the scope of the present invention to use one or more fusion segments. Fusion segments can be joined to amino and/or carboxyl termini of the EG307 or EG1117-containing domain of the polypeptide. Linkages between fusion segments and EG307 or EG1117-containing domains of fusion polypeptides can be susceptible to cleavage in order to enable straightforward recovery of the EG307 or EG 1117-containing domains of such polypeptides. Fusion polypeptides are produced in some embodiments by culturing a recombinant cell transformed with a fusion polynucleotide that encodes a polypeptide including the fusion segment attached to either the carboxyl and/or amino terminal end of a EG307 or EG1117-containing domain.

Some fusion segments for use in the present invention include a glutathione binding domain; a metal binding domain, such as a poly-histidine segment capable of binding to a divalent metal ion; an immunoglobulin binding domain, such as Polypeptide A, Polypeptide G, T cell, B cell, Fc receptor or complement polypeptide antibody-binding domains; a sugar binding domain such as a maltose binding domain from a maltose binding polypeptide; and/or a “tag” domain (e.g., at least a portion of β-galactosidase, a strep tag peptide, other domains that can be purified using compounds that bind to the domain, such as monoclonal antibodies). Other fusion segments include metal binding domains, such as a poly-histidine segment; a maltose binding domain; a strep tag peptide.

As used herein, “at least a portion” of a polynucleotide or polypeptide means a portion having the minimal size characteristics of such sequences, as described above, or any larger fragment of the full length molecule, up to and including the full length molecule. For example, a portion of a polynucleotide may be 12 nucleotides, 13 nucleotides, 14 nucleotides, 15 nucleotides, and so on, going up to the full length polynucleotide. Similarly, a portion of a polypeptide may be 4 amino acids, 5 amino acids, 6 amino acids, 7 amino acids, and so on, going up to the full length polypeptide. The length of the portion to be used will depend on the particular application. As discussed above, a portion of a polynucleotide useful as hybridization probe may be as short as 12 nucleotides. A portion of a polypeptide useful as an epitope may be as short as 4 amino acids. A portion of a polypeptide that performs the function of the full-length polypeptide would generally be longer than 4 amino acids.

Other plant EG307 or EG1117 polypeptides of the present invention are polypeptides that include but are not limited to the encoded polypeptides, full-length polypeptides, processed polypeptides, fusion polypeptides and multivalent polypeptides thereof as well as polypeptides that are truncated homologues of polypeptides that include at least portions of the aforementioned SEQ ID NOs.

The named sequences of the present invention are discussed in Table II. Table II shows the sequence identification number, the gene, the species from which it was isolated, a description of the sequence, the priority application from which it originated, and its original sequence identification number in the priority application. All named sequences in the present application are yield-related genes and are capable of altering the yield of a plant, e.g., the named sequences are capable of increasing the yield of a plant and/or decreasing the yield of a plant. Methods to assess yield are described elsewhere herein.

TABLE II

| SEQ | | | | USSN | SEQ ID NO IN |
|---|---|---|---|---|---|
| ID | | | | ORIGINATED | PRIORITY |
| NO | GENE | SPECIES | DESCRIPTION | FROM | APPLICATION |
| 41 | EG307 | Z. mays mays | CDS nonelite corn allele I | USSN 60/774,939 | 1 |
| 42 | EG307 | Z. mays mays | Cds | USSN 60/774,939 | |
| 43 | EG307 | Z. mays mays | Protein | USSN 60/774,939 | |
| 44 | EG307 | Z. mays mays | CDS nonelite corn allele II | USSN 60/774,939 | 2 |
| 45 | EG307 | Z. mays mays | CDS | USSN 60/774,939 | |
| 46 | EG307 | Z. mays mays | Protein | USSN 60/774,939 | |
| 47 | EG307 | Z. mays mays\ | CDS nonelite corn allele III | USSN 60/774,939 | 3 |
| 48 | EG307 | Z. mays mays | CDS | USSN 60/774,939 | |
| 49 | EG307 | Z. mays mays | protein | USSN 60/774,939 | |
| 50 | EG307 | Z. mays mays | CDS nonelite corn allele IV | USSN 60/774,939 | 4 |
| 51 | EG307 | Z. mays mays | CDS | USSN 60/774,939 | |
| 52 | EG307 | Z. mays mays | Protein | USSN 60/774,939 | |
| 53 | EG307 | Z. mays mays | CDS nonelite corn allele V | USSN 60/774,939 | 5 |
| 54 | EG307 | Z. mays mays | CDS | USSN 60/774,939 | |
| 55 | EG307 | Z. mays mays | protein | USSN 60/774,939 | |
| 56 | EG307 | Z. mays mays | CDS nonelite corn allele VI | USSN 60/774,939 | 6 |
| 57 | EG307 | Z. mays mays | CDS | USSN 60/774,939 | |
| 58 | EG307 | Z. mays mays | protein | USSN 60/774,939 | |
| 59 | EG307 | Z. mays mays | CDS nonelite corn allele VII | USSN 60/774,939 | 7 |
| 60 | EG307 | Z. mays mays | CDS | USSN 60/774,939 | |
| 61 | EG307 | Z. mays mays | protein | USSN 60/774,939 | |
| 62 | EG307 | Z. mays mays | CDS nonelite corn allele VIII | USSN 60/774,939 | 8 |
| 63 | EG307 | Z. mays mays | CDS | USSN 60/774,939 | |
| 64 | EG307 | Z. mays mays | protein | USSN 60/774,939 | |
| 65 | EG307 | Z. mays mays | CDS nonelite corn allele IX | USSN 60/774,939 | 9 |
| 66 | EG307 | Z. mays mays | CDS | USSN 60/774,939 | |
| 67 | EG307 | Z. mays mays | protein | USSN 60/774,939 | |
| 68 | EG307 | Z. mays mays | CDS nonelite corn allele X | USSN 60/774,939 | 10 |
| 69 | EG307 | Z. mays mays | CDS | USSN 60/774,939 | |
| 70 | EG307 | Z. mays mays | protein | USSN 60/774,939 | |
| 71 | EG307 | Z. mays mays | CDS elite corn allele I + UTR | USSN 60/774,939 | 11 |
| 72 | EG307 | Z. mays mays | CDS coding | USSN 60/774,939 | |
| 73 | EG307 | Z. mays mays | protein | USSN 60/774,939 | |
| 74 | EG307 | Z. mays mays | CDS elite corn allele II + UTR | USSN 60/774,939 | 12 |
| 75 | EG307 | Z. mays mays | CDS coding | USSN 60/774,939 | |
| 76 | EG307 | Z. mays mays | protein | USSN 60/774,939 | |
| 77 | EG9703 | Z. mays mays | CDS corn partial EST | USSN 60/774,939 | 13 |
| 78 | EG307 | O. saliva | Forward primer | USSN 60/666,511 | 14 |
| 79 | EG307 | O. saliva | Reverse primer | USSN 60/666,511 | 15 |
| 80 | EG307 | O. saliva | probe | USSN 60/666,511 | 16 |
| 81 | EG307 | O. saliva | probe | USSN 60/666,511 | 17 |
| 82 | EG307 | O. saliva | Forward primer | USSN 60/666,511 | 18 |
| 83 | EG307 | O. saliva | Reverse primer | USSN 60/666,511 | 19 |
| 84 | EG307 | O. saliva | probe | USSN 60/666,511 | 20 |
| 85 | EG307 | O. saliva | probe | USSN 60/666,511 | 21 |
| 86 | EG1117 | O. saliva | Forward primer | USSN 60/666,511 | 22 |
| 87 | EG1117 | O. saliva | Reverse primer | USSN 60/666,511 | 23 |
| 88 | EG1117 | O. saliva | probe | USSN 60/666,511 | 24 |
| 89 | EG1117 | O. saliva | probe | USSN 60/666,511 | 25 |
| 90 | EG1117 | O. saliva | Forward primer | USSN 60/666,511 | 26 |
| 91 | EG1117 | O. saliva | Reverse primer | USSN 60/666,511 | 27 |
| 92 | EG1117 | O. saliva | probe | USSN 60/666,511 | 28 |
| 93 | EG1117 | O. saliva | probe | USSN 60/666,511 | 29 |
| 94 | EG1117 | Z. mays mays | Full sequence including UTR | USSN 60/666,511 | 2 |
| 95 | EG1117 | Z. mays mays | CDS | USSN 60/666,511 | 1 |
| 96 | EG1117 | Z. mays mays | protein | USSN 60/666,511 | 30 |
| 97 | EG1117 | S. bicolor | 5′ end | USSN 60/666,511 | 3 |
| 98 | EG1117 | S. bicolor | 3′ end | USSN 60/666,511 | 4 |
| 99 | EG1117 | S. bicolor | protein | USSN 60/666,511 | 33 |
| 100 | EG1117 | S. officinarum | 5′ end contains UTR EST | USSN 60/666,511 | 5 |
| 101 | EG1117 | S. officinarum | CDS | USSN 60/666,511 | |
| 102 | EG1117 | S. officinarum | protein | USSN 60/666,511 | |
| 103 | EG1117 | S. officinarum | 3′ end | USSN 60/666,511 | 6 |
| 104 | EG1117 | S. officinarum | protein | USSN 60/666,511 | 35 |
| 105 | EG1117 | T. aestivum | Cluster Y | USSN 60/666,511 | 7 |
| 106 | EG1117 | T. aestivum | protein | USSN 60/666,511 | 36 |
| 107 | EG1117 | T. aestivum | Cluster x | USSN 60/666,511 | 8 |
| 108 | EG1117 | T. aestivum | protein | USSN 60/666,511 | 37 |
| 109 | EG1117 | T. aestivum | Cluster z | USSN 60/666,511 | 9 |
| 110 | EG1117 | T. aestivum | protein | USSN 60/666,511 | 38 |
| 111 | EG1117 | H. vulgare | Copy D with 3′ UTR | USSN 60/666,511 | 10 |
| 112 | EG1117 | H. vulgare | protein | USSN 60/666,511 | |
| 113 | EG1117 | H. vulgare | Copy A with 3′ UTR | USSN 60/666,511 | 11 |
| 114 | EG1117 | H. vulgare | protein | USSN 60/666,511 | |
| 115 | EG307 | H. vulgare | Coding sequence | USSN 60/666,511 | 12 |
| 116 | EG307 | H. vulgare | Protein | USSN 60/666,511 | 39 |
| 117 | EG307 | T. aestivum | Partial coding sequence | USSN 60/666,511 | 13 |
| 118 | EG307 | T. aestivum | Protein | USSN 60/666,511 | 40 |
| 119 | EG307 | S. bicolor | CDS | | |
| 120 | EG307 | S. bicolor | protein | | |

With regard to EG307 or EG1117, some recombinant cells are plant cells. By “plant cell” is meant any self-propagating cell bounded by a semi-permeable membrane and containing a plastid. Such a cell also requires a cell wall if further propagation is desired. Plant cell, as used herein includes, without limitation, algae, cyanobacteria, seeds, suspension cultures, embryos, meristematic regions, callus tissue, leaves, roots, shoots, gametophytes, sporophytes, pollen, and microspores. Characteristics of recombinant cells and transgenic plants and suitable methods are described in WO 03/062382, as well as U.S. Pat. No. 6,040,497, both of which are incorporated by reference in their entireties. For example, expression of genes in corn is known in the art and appropriate promoters are known and may be selected by the knowledgeable artesan. For example, plant expression vectors may be constructed using known maize expression vectors, such as those which can be obtained from Rhone Poulenc Agrochimie. Methods to construct the expression constructs and transformation vectors include standard in vitro genetic recombination and manipulation. See, for example, the techniques described in Weissbach and Weissbach, 1988, Methods For Plant Molecular Biology, Academic Press, Chapters 26-28. The transformation vectors of the invention may be developed from any plant transformation vector known in the art including, but are not limited to, the well-known family of Ti plasmids from Agrobacterium and derivatives thereof, including both integrative and binary vectors, and including but not limited to pBIB-KAN, pGA471, pEND4K, pGV38SO, and pMONSOS. Also included are DNA and RNA plant viruses, including but not limited to CaMV, geminiviruses, tobacco mosaic virus, and derivatives engineered therefrom, any of which can effectively serve as vectors to transfer a coding sequence, or functional equivalent thereof, with associated regulatory elements, into plant cells and/or autonomously maintain the transferred sequence. In addition, transposable elements may be utilized in conjunction with any vector to transfer the coding sequence and regulatory sequence into a plant cell.

To aid in the selection of transformants and transfectants, the transformation vectors may preferably be modified to comprise a coding sequence for a reporter gene product or selectable marker. Such a coding sequence for a reporter or selectable marker should preferably be in operative association with the regulatory element coding sequence described supra.

Reporter genes which may be useful in the invention include but are not limited to the ′3-glucuronidase (GUS) gene (Jefferson et al., Proc. Natl. Acad. Sci. USA, 83:8447 (1986)), and the luciferase gene (Ow et al., Science 234:856 (1986)). Coding sequences that encode selectable markers which may be useful in the invention include but are not limited to those sequences that encode gene products conferring resistance to antibiotics, anti-metabolites or herbicides, including but not limited to kanamycin, hygromycin, streptomycin, phosphinothricin, gentamicin, methotrexate, glyphosate and sulfonylurea herbicides, and include but are not limited to coding sequences that encode enzymes such as neomycin phosphotransferase II (NPTII), chloramphenicol acetyltransferase (CAT), and hygromycin phosphotransferase I (HPT, HYG).

A variety of plant expression systems may be utilized to express the coding sequence or its functional equivalent. Particular plant species may be selected from any dicotyledonous, monocotyledonous species, gymnospermous, lower vascular or non-vascular plant, including any cereal crop or other agriculturally important crop. Such plants include, but are not limited to, alfalfa, Arabidopsis, asparagus, wheat, sugarcane, pearl millet, sorghum, barley, cabbage, carrot, celery, corn, cotton, cucumber, flax, lettuce, oil seed rape, pear, peas, petunia, poplar, potato, rice, beet, sunflower, tobacco, tomato, wheat and white clover. Methods by which plants may be transformed or transfected are well-known to those skilled in the art. See, for example, Plant Biotechnology, 1989, Kung & Arntzen, eds., Butterworth Publishers, ch. 1, 2. Examples of transformation methods which may be effectively used in the invention include but are not limited to Agrobacterium-mediated transformation of leaf discs or other plant tissues, microinjection of DNA directly into plant cells, electroporation of DNA into plant cell protoplasts, liposome or spheroplast fusion, microprojectile bombardment, and the transfection of plant cells or tissues with appropriately engineered plant viruses. Plant tissue culture procedures necessary to practice the invention are well-known to those skilled in the art. See, for example, Dixon, 1985, Plant Cell Culture: A Practical Approach, IRL Press. Those tissue culture procedures that may be used effectively to practice the invention include the production and culture of plant protoplasts and cell suspensions, sterile culture propagation of leaf discs or other plant tissues on media containing engineered strains of transforming agents such as, for example, Agrobacterium or plant virus strains and the regeneration of whole transformed plants from protoplasts, cell suspensions and callus tissues. The invention may be practiced by transforming or transfecting a plant or plant cell with a transformation vector containing an expression construct comprising a coding sequence for the sequence and selecting for transformants or transfectants that express the sequence. Transformed or transfected plant cells and tissues may be selected by techniques well-known to those of skill in the art, including but not limited to detecting reporter gene products or selecting based on the presence of one of the selectable markers described supra. The transformed or transfected plant cells or tissues are then grown and whole plants regenerated therefrom. Integration and maintenance of the coding sequence in the plant genome can be confirmed by standard techniques, e.g., by Southern hybridization analysis, PCR analysis, including reverse transcriptase-PCR (RT-PCR) or immunological assays for the expected protein products. Once such a plant transformant or transfectant is identified, a non-limiting embodiment of the invention involves the clonal expansion and use of that transformant or transfectant in the production of a sequence.

Regulatory elements that may be used in the expression constructs include promoters which may be either heterologous or homologous to the plant cell. The promoter may be a plant promoter or a non-plant promoter which is capable of driving high levels transcription of a linked sequence in plant cells and plants. Non-limiting examples of plant promoters that may be used effectively in practicing the invention include cauliflower mosaic virus (CaMV) 19S or 35S, rbcS, the promoter for the chlorophyll a/b binding protein, AdhI, NOS and HMG2, or modifications or derivatives thereof. The promoter may be either constitutive or inducible. For example, and not by way of limitation, an inducible promoter can be a promoter that promotes expression or increased expression of the polynucleotides of the present invention after mechanical gene activation (MGA) of the plant, plant tissue or plant cell. One non-limiting example of such an MGA-inducible plant promoter is MeGA.

The expression constructs can be additionally modified according to methods known to those skilled in the art to enhance or optimize heterologous gene expression in plants and plant cells. Such modifications include but are not limited to mutating DNA regulatory elements to increase promoter strength or to alter the coding sequence itself. Other modifications include deleting intron sequences or excess non-coding sequences from the 5′ and/or 3′ ends of the coding sequence in order to minimize sequence- or distance-associated negative effects on expression of proteins, e.g., by minimizing or eliminating message destabilizing sequences.

The expression constructs may be further modified according to methods known to those skilled in the art to add, remove, or otherwise modify peptide signal sequences to alter signal peptide cleavage or to increase or change the targeting of the expressed polypeptides through the plant endomembrane system. For example, but not by way of limitation, the expression construct can be specifically engineered to target the polypeptide for secretion, or vacuolar localization, or retention in the endoplasmic reticulum (ER).

The present invention also includes isolated antibodies capable of selectively binding to an EG307 or EG1117 polypeptide of the present invention or to a mimetope thereof. Characteristics of recombinant cells and transgenic plants, and suitable methods are described in WO 03/062382.

The present invention also includes plant cells, which comprise heterologous DNA encoding an EG1117 or EG307 polypeptide. Such polypeptides are capable of altering the yield of a plant. For example, most preferably the polypeptide is capable of increasing the yield of a plant, and less preferably the polypeptide is capable of decreasing the yield of a plant. The plant cells include the polypeptides of the present invention as described elsewhere herein. Additionally, the present invention includes a propagation material of a transgenic plant comprising the above-described transgenic plant cell.

The present invention also includes transgenic plants containing heterologous DNA which encodes an EG1117 or EG307 polypeptide that is expressed in plant tissue. Such polypeptides are capable of altering the yield of a plant. The transgenic plants include the polypeptides of the present invention as described elsewhere herein.

The present invention also includes an isolated polynucleotide which includes a promoter operably linked to a polynucleotide that encodes an EG1117 or EG307 polypeptide in plant tissue. Such polypeptides are capable of altering the yield of a plant. The transgenic plants include the polypeptides of the present invention as described elsewhere herein The polynucleotide can be a recombinant polynucleotide, and may include any promoter, including a promoter native to an EG1117 or EG307 gene.

The present invention also includes a transfected host cell comprising a host cell transfected with a construct comprising a promoter, enhancer or intron polynucleotide from an EG1117 or EG307 polynucleotide or any combination thereof, operably linked to a polynucleotide encoding a reporter protein. Such constructs are capable of altering the yield of a plant. The transfected host cells comprise the polypeptides of the present invention as described elsewhere herein.

The present invention also includes a recombinant vector, which includes at least one plant EG307 or EG1117 polynucleotide of the present invention, inserted into any vector capable of delivering the polynucleotide into a host cell. Characteristics of recombinant molecules and suitable methods are described in WO 03/062382. Suitable polynucleotides to include in recombinant vectors of the present invention are as disclosed herein for suitable plant EG307 or EG1117 polynucleotides per se. Polynucleotides to include in recombinant vectors, and particularly in recombinant molecules, of the present invention include the EG307 and EG1117 polynucleotides of the present invention.

As used herein, stringent hybridization conditions refer to standard hybridization conditions under which polynucleotides, including oligonucleotides, are used to identify molecules having similar nucleic acid sequences. Such standard conditions are disclosed, for example, in Sambrook et al., MOLECULAR CLONING: A LABORATORY MANUAL, Cold Spring Harbor Labs Press, 1989. Examples of such conditions are provided in the Examples section of the present application.

As used herein, a corn EG307 or EG1117gene includes all nucleic acid sequences related to a natural corn EG307 or EG1117gene such as regulatory regions that control production of the corn EG307 or EG1117 polypeptide encoded by that gene (such as, but not limited to, transcription, translation or post-translation control regions) as well as the coding region itself. In one embodiment, an corn EG307 or EG1117gene includes the nucleic acid sequence SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; SEQ ID NO:93 (as well as other sequences presented herein).

In another embodiment, a corn EG307 or EG1117gene can be an allelic variant that includes a similar but not identical sequence to an EG307 or EG1117 of the present invention, is a locus (or loci) in the genome whose activity is concerned with the same biochemical or developmental processes, and/or a gene that that occurs at essentially the same locus as the genes including an EG307 or EG1117 gene of the present invention, but which, due to natural variations caused by, for example, mutation or recombination, has a similar but not identical sequence. Because genomes can undergo rearrangement, the physical arrangement of alleles is not always the same. Allelic variants typically encode polypeptides having similar activity to that of the polypeptide encoded by the gene to which they are being compared. Allelic variants can also comprise alterations in the 5′ or 3′ untranslated regions of the gene (e.g., in regulatory control regions). Allelic variants are well known to those skilled in the art and would be expected to be found within a given cultivar or strain since the genome is multiploid and/or among a population comprising two or more cultivars or strains. An allele can be defined as a EG1117 or EG307 polynucleotide sequence having at least one nucleotide change compared to a second EG1117 or EG307 polynucleotide sequence.

As such, the minimal size of a polynucleotide used to encode an EG307 or EG1117 polypeptide homologue of the present invention is from about 12 to about 18 nucleotides in length. There is no limit, other than a practical limit, on the maximal size of such a polynucleotide in that the polynucleotide can include a portion of a gene, an entire gene, or multiple genes, or portions thereof. Similarly, the minimal size of an EG307 or EG1117 polypeptide homologue of the present invention is from about 4 to about 6 amino acids in length, with the desired sizes depending on whether a full-length, fusion, multivalent, or functional portions of such polypeptides are desired. In some embodiments, the polypeptide is at least 30 amino acids in length.

As used herein, a EG307 or EG1117gene includes all nucleic acid sequences related to a natural EG307 or EG1117gene such as regulatory regions that control production of the EG307 or EG1117 polypeptide encoded by that gene (such as, but not limited to, transcription, translation or post-translation control regions) as well as the coding region itself. In one embodiment, an EG307 or EG1117 gene includes the EG307 or EG1117 polynucleotides of the present invention. In another embodiment, a corn EG307 or EG1117gene can be an allelic variant that includes a similar but not identical sequence to the EG307 or EG1117 polynucleotides of the present invention.

As used herein, an EG307 or EG1117gene includes all nucleic acid sequences related to a natural EG307 or EG1117 gene such as regulatory regions that control production of the EG307 or EG1117 polypeptide encoded by that gene (such as, but not limited to, transcription, translation or post-translation control regions) as well as the coding region itself. An EG307 or EG1117 gene may preferably include the EG307 or EG1117 polynucleotides of the present invention. Additional objects, advantages, and novel features of this invention will become apparent to those skilled in the art upon examination of the following examples thereof, which are not intended to be limiting.

### Example 1

### Confirming Validation of Yield Candidate Genes: Association Analysis

As described in Example 17 of WO 03/062382, association analysis involves sequencing each candidate gene in a large number of well-characterized rice strains to learn if the genes are associated with known traits. In addition to rice lines analyzed as described in Example 17, 44 well-characterized rice strains (see Table 1) were analyzed for EG307 and EG1117 allele. As was found previously, the derived, positively-selected allele of each of EG307 and EG1117 correlated with higher grain weight in these 44 rice lines. Using a chi-square test for association, we found the association between allele (genotype) and phenotype was significant with 2 degrees of freedom, P<0.0001. The pattern that is observed from the following table shows that EG307 does influence yield, i.e., is a yield related gene capable of increasing yield in a plant.

TABLE III
Genotyping of Rice Accessions Sorted by Grain Weight

| | 1000-grain | | | | |
|---|---|---|---|---|---|
| Accession | weight | 307 A | 1117 A | 307 D | 1117 D |
| AC 27 | 45.97 | | | X | X |
| CSELJAJ | 43.25 | | | X | X |
| Kokoku Mochi | 40.55 | | | X | X |
| Razza 77 | 38.64 | | | X | X |
| Arborio | 38.31 | | | X | X |
| Baldo | 37.44 | | | X | X |
| Vary Voto 277 | 37.17 | | | X | X |
| Uz Rosz | 36.5 | | | X | X |
| Sesia | 36.49 | | | X | X |
| Fortuna | 35.73 | | | X | X |
| Stirpe 136 | 33.18 | | | X | X |
| Vialone | 33.13 | | | X | X |
| Azucena | 32.08 | | | X | X |
| Caloro | 28.73 | | | X | X |
| 79 | 27.49 | X | X | | |
| IR8 | 26.78 | X | X | | |
| IR24 | 26.30 | X | X | | |
| PR103 | 26.17 | X | X | | |
| Early Prolific | 25.87 | | | X | X |
| Texas Patna | 24.14 | | | X | X |
| Dalila | 24.28 | X | X | | |
| Family 24 | 24.13 | | | X | X |
| TOTO | 23.97 | X | X | | |
| Sathri Sufaid | 23.95 | X | X | | |
| Zenith | 23.93 | | | X | X |
| CR94-13 | 23.64 | X | X | | |
| Lady Wright | 23.48 | | | X | X |
| Ccntury Patna 231 | 23.00 | | | X | X |
| Desi Sathri Ratti | 22.71 | X | X | | |
| IR36 | 22.50 | X | X | | |
| Palawan | 22.40 | | | X | X |
| C22 | 20.04 | X | X | | |
| IR20 | 19.24 | X | X | | |
| Sinampaga | 18.13 | | | X | X |
| IR40 | 17.80 | X | X | | |
| Amber 43 | 15.30 | X | X | | |
| Sigadis | 14.55 | X | X | | |
| BR51-46-5 | 10.90 | X | X | | |
| Ngoat | 9.57 | X | X | | |
| Jira Sahai | 9.05 | X | X | | |
| BR51-91-6 | 9.04 | X | X | | |
| T88 | 8.0 | X | X | | |
| BR52-8-1 | 6.89 | X | X | | |
| IR1545-339-2-2 | 3.37 | X | X | | |

307 A = EG307 ancestral allele;
307 D = EG307 derived (adapted) allele
1117 A = EG1117 ancestral allele;
1117 D = EG1117 derived (adapted) allele

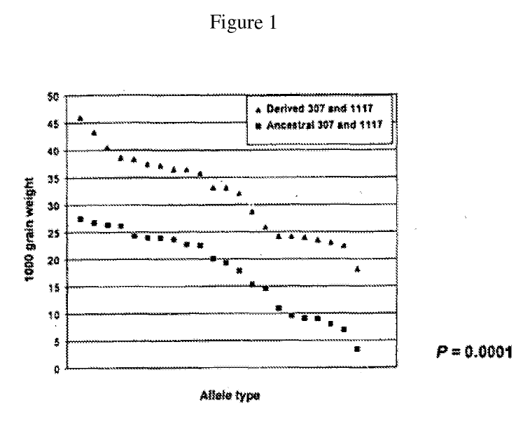

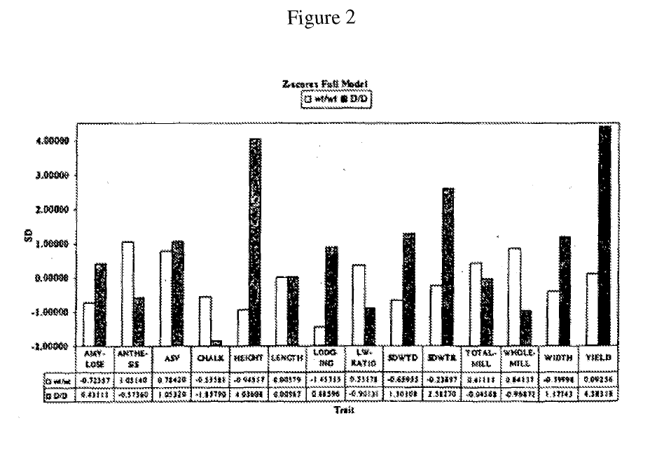

FIG. 1 is a plot showing data in Table III showing the range of grain weights correlated with either the ancestral (square) or derived (triangle) allele for either EG307 or EG1117. In FIG. 2, phenotypic data were converted to Z scores, values expressing to what extent a trait is affected by a particular genotype. The Z score indicates how far and in what direction a traitdeviates from the trait's distribution's mean, expressed in units of the trait's distribution's standard deviation (SD). Z scores greater than 1 SD indicate an effect of the allele and the trait. The greater the Z score, the greater the effect. The Z score for yield was 4 SD, a very pronounced effect.

An additional 104 well-characterized rice lines and hybrids were then genotyped using a more high-throughput method. The ancestral allele for each of EG307 and EG1117 can be distinguished from the derived (adapted) allele by examining the nucleotides at a few key positions. Thus, instead of genotyping by sequencing the entire coding sequence, we genotyped by analyzing the nucleotide present in a few key positions. Primers were designed that would produce a small (no greater than 200 bp product, preferable 100-150 bp) PCR product surrounding the position to be analyzed. Next, a probe was designed that would span the position, having the position to be analyzed as close to the center of the probe as possible. The probe was as short as possible without being shorter than 12 bps. Additionally, the probes were designed such that they had a melting temperature (Tm) in the range 65 to 67° C. Two probes were designed for each set of primers, one for each position to be analyzed. Using ABI MGB quencher technology, higher Tms can be used for the probes than are used for the actual PCR product itself. Each probe was synthesized incorporating a different fluorescent tag (either VIC or FAM).

A primer/probe mix was made that included one set of forward and reverse primers and both probes (see Table IV). A Biotage Rotor-Gene 3000 RT PCR system was used according to manufacturer's protocols for genotyping. For lines or hybrids that are homozygous for either the ancestral or the derived allele, only the probe that is specific for the nucleotide corresponding to either the ancestral or the derived allele will attach to the product as it is made in the thermocycling reaction and consequently fluoresce. When both are present (as in a heterozygote), both fluorescent dyes are seen in the PCR reaction.

TABLE IV
Rice Genotyping Primers and Probes

| EG307 | Position: 623 2041-(76-77) | |
|---|---|---|
| | Forward Primer | |
| | CGAAATGATGGTGAGAACAGCAT | (SEQ ID No. 78) |
| | Reverse Primer | |
| | TCGACTCTTGGCATGACTTTTG | (SEQ ID No. 79) |
| | Probes | |
| | CAGTAC [symbol] GAAACAA | (SEQ ID No. 80) |
| | CAGTAC [symbol] GAAACAAGG | (SEQ ID No. 81) |
| EG307 | Position: 329 2014-(74-75) | |
| | Forward Primer | |
| | GGAACCTGGTGAGCAATTGG | (SEQ ID No. 82) |
| | Reverse Primer | |
| | GGACTGGGTAACACAACCTTTCTT | (SEQ ID No. 83) |
| | Probes | |
| | CAGACAG [symbol] GCATGGC | (SEQ ID No. 84) |
| | CAGACAG [symbol] GCATGGC | (SEQ ID No. 85) |
| EG1117 | Position: 2 2014-(72-73) | |
| | Forward | |
| | TGTCATCAGTGTCATCATCTGGATT | (SEQ ID No. 86) |
| | Reverse | |
| | CCCTTCCAGTGAACTTTCTAGCTATT | (SEQ ID No. 87) |
| | Probes | |
| | CCGTTTTATG [symbol] CCGTGTG | (SEQ ID No. 88) |
| | CCGTTTTATG [symbol] CCGTGTG | (SEQ ID No. 89) |
| EG1117 | Position: 1 2012-93 | |
| | Forward | |
| | CCATTTGGGCCACTACTATTA | (SEQ ID No. 90) |
| | Reverse | |
| | TCATTGTCCCTCCTGCATCC | (SEQ ID No. 91) |
| | Probes | |
| | ATGCTCA [symbol] AACTCT | (SEQ ID No. 92) |
| | ATGCTCA [symbol] AACTCTT | (SEQ ID No. 93) |

Using a single-factor additive statistical model corrected for line effects, we analyzed the effect of genotype (homozygous ancestral or homozygous derived alleles for each of EG307 and EG1117). Six estimates were greater than one standard deviation (a major gene effect) with the most pronounced effects in decreasing order on: yield, plant height, rough grain weight (sdwt1000-rough), dehulled grain weight (sdwt1000-dehulled), width, and AS. Less pronounced estimated plus effects were on lodging, amylase and length. There was one major estimated negative effect for the derived alleles of both genes on the chalk trait. Chalk is generally an undesirable feature of rice, although it can be desirable in certain specialized types of rice. Chalk results from formation of misshaped starch granules that pack differently than properly shaped starch granules leaving air spaces between them. The domesticated (derived) alleles of EG307 and EG1117 correlate with less chalk.

We then calculated R², the proportion of variation explained by the single-factor additive model corrected for line effects. For the major plus effects, R² ranged from 47% for yield, 35% for height, 35% for dehulled grain weight, 18% for width, 15% for ASV (alkaline spreading value, when combined with % amylase, yields the starch index), 11% for rough grain weight, and 19% for chalk.

### Example 2

### Identification of EG307 and EG1117 in Wheat, Barley, Sorghum, and Sugar Cane

Searching the wheat, barley, sorghum, and sugar cane genome sequences in GenBank by BLAST using rice EG1117 sequences identified at least four wheat ESTs (including accession numbers CK203588, CK203242, BE444-456, and BJ481258), several barley ESTs (including accession numbers BJ478960 and BJ481259), 4 sorghum ESTs (including accession numbers CA200440, BG948036, BG947743, and BM327663), and 5 sugar cane ESTs (accession numbers CA284889, CA102585, CA200440, CA218123, and CA106550) which appear to be homologous. Primers were designed by standard methods that allowed successful amplification of the wheat, barley, sorghum, and sugar cane homologs. Sequences of wheat, barley, sorghum, and sugarcane homologs are provided herein. Modern bread wheat is a hexaploid, consisting of three genomes, so we expected to see three copies of EG1117 and EG307. In the case of EG1117 we know there are at least three expressed copies.

Searching the wheat and barley genome sequences in GenBank by BLAST using rice EG307 sequences identified a number of wheat ESTs (including accession numbers CD898159, BE496848, BF484251, CA595746, CA730688, and CV772418) and nine barley ESTs (including accession numbers BI958390, CA026456, BQ467189, CA002341, BE558500, BQ466901, BU996029, CA014071, CB867549) which appear to be homologous. Primers were designed by standard methods that allowed successful amplification of the wheat and barley homologs. Sequences are provided herein.

### Example 3

### Association Analysis in Maize

Using sequence data from Oryza and maize ESTs primers were designed and EG307 and EG1117 were PCR amplified in ancestral corn (teosinte), three corn landraces, and 6 commercially available elite hybrids. The alleles of EG307 found clustered into two groups. One group of closely related alleles, including allele A (SEQ ID NO. 71) and allele B (SEQ ID NO. 74) were found only in the six elite hybrids. The other group of closely related alleles, including allele I (SEQ ID NO. 41), allele II (SEQ ID NO. 44), allele III (SEQ ID NO. 47), allele IV (SEQ ID NO. 50), allele V (SEQ ID NO. 53), allele VI (SEQ ID NO. 56), allele VII (SEQ ID NO. 59), allele VIII (SEQ ID NO. 62), allele IX (SEQ ID NO. 65), and allele X (SEQ ID NO. 68) were found only in the lower yielding ancestral corn or landraces.

It is noted that a number of sequences, such as ESTs, existing in the public domain. Some ESTs may have areas of high identity with the polynucleotides disclosed herein in areas of overlap with the polynucleotides of the present invention. In other words, there could potentially be regions of lower identity between a polynucleotide of the present invention and a sequence in the public domain, such as an EST, where the sequence or EST does not overlap with the polynucleotide of the present invention, and regions of higher identity where that EST overlaps with a polynucleotide of the present invention. Regions of lower identity may be specified by the inventors, these regions will comprise areas of the named SEQ ID NO: that do not have an overlap with a sequence or EST in the public domain, and these regions of lower identity will have a percent identity of at least about 50%, at least about 55%, at least about 58%, at least about 60%, at least about 61%, at least about 62%, at least about 63%, at least about 64%, at least about 65%, at least about 66%, at least about 67%, at least about 68%, or at least about 70%, at least about 75%, at least about 80%, at least about 85%, or at least about 90% to a named SEQ ID NO: herein. These regions may be claimed separately by calling out their position in the SEQ ID NO:, for example, a region may be identified as follows: nucleotides 1 to 144 of SEQ ID NO:105.

### Example 4

### Using Genotype as Markers for Marker Assisted Selection or Marker Assisted Breeding

In crosses using landrace lines to try to bring better drought resistance or pest resistance into an elite hybrid, but not lose yield, seedlings from such cross are screened and only those seedlings that contain the best allele of EG 307 or EG1117 are selected. In crosses of a lower yielding inbred and a higher yielding inbred—seedlings from such cross are screened and only those seedlings that contain the best allele of EG307 or EG1117 are selected.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for identifying a polynucleotide sequence that is associated with yield in plant, comprising the steps of:
a) comparing at least a portion of plant polynucleotide sequence with at least one polynucleotide selected from the group consisting of
i) SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; SEQ ID NO:93; and
ii) a polynucleotide having at least about 95% sequence identity to a polynucleotide in a); and
b) identifying at least one polynucleotide sequence in the plant that contains at least one nucleotide change as compared to a polynucleotide of a), wherein said identified polynucleotide sequence is associated with yield in a plant.

2. (canceled)

3. The method of claim 1, wherein the plant polynucleotide sequence is genomic DNA.

4. The method of claim 1, wherein the plant polynucleotide sequence is cDNA.

5-6. (canceled)

7. The method of claim 1, wherein the plant is selected from the group consisting of Zea mays mays, Oryza sativa, Triticum aestivum, Hordeum vulgare, Saccharum officinarum, Sorghum bicolor, and Pennisetum typhoides.

8. The method of claim 1, wherein the plant is selected from the group consisting of a wild ancestor plant for a domesticated plant selected from the group consisting of Zea mays mays, Oryza sativa, Triticum aestivum, Hordeum vulgare, Saccharum officinarum, Sorghum bicolor, and Pennisetum typhoides.

9. The method of claim 8, wherein the plant is Oryza rufipogon or teosinte.

10. An isolated polynucleotide selected from the group consisting of: a) a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; SEQ ID NO:93; and b) a polynucleotide having at least about 95% homology to a polynucleotide of a), and confers substantially the same yield as a polynucleotide of a).

11. An isolated polypeptide selected from the group consisting of:
a) a polypeptide encoded by a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; and SEQ ID NO:113;
b) a polypeptide encoded by a polynucleotide having at least about 95% sequence identity to a polynucleotide in a) and confers substantially the same yield as a polynucleotide of a);
c) a polypeptide comprising SEQ ID NO:73; SEQ ID NO:76; SEQ ID NO:43; SEQ ID NO:46; SEQ ID NO:49; SEQ ID NO:52; SEQ ID NO:55; SEQ ID NO:58; SEQ ID NO:61; SEQ ID NO:64; SEQ ID NO:67; SEQ ID NO:70; SEQ ID NO:118; SEQ ID NO:120; SEQ ID NO:116; SEQ ID NO:96; SEQ ID NO:99; SEQ ID NO:102; SEQ ID NO:104; SEQ ID NO:106; SEQ ID NO:108; SEQ ID NO:110; SEQ ID NO:112; and SEQ ID NO:114; and
d) a polypeptide having at least about 95% sequence identity to a polypeptide of c) and confers substantially the same yield as a polypeptide of c).

12. Plant cells, comprising heterologous DNA encoding an EG1117 or EG307 polypeptide wherein said polypeptide is capable of increasing the yield of a plant, wherein said polypeptide is selected from the group consisting of:
a) a polypeptide encoded by a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; and SEQ ID NO:113;
b) a polypeptide encoded by a polynucleotide having at least about 95% sequence identity to a polynucleotide in a);
c) a polypeptide comprising SEQ ID NO:73; SEQ ID NO:76; SEQ ID NO:43; SEQ ID NO:46; SEQ ID NO:49; SEQ ID NO:52; SEQ ID NO:55; SEQ ID NO:58; SEQ ID NO:61; SEQ ID NO:64; SEQ ID NO:67; SEQ ID NO:70; SEQ ID NO:118; SEQ ID NO:120; SEQ ID NO:116; SEQ ID NO:96; SEQ ID NO:99; SEQ ID NO:102; SEQ ID NO:104; SEQ ID NO:106; SEQ ID NO:108; SEQ ID NO:110; SEQ ID NO:112; and SEQ ID NO:114; and
d) a polypeptide having at least about 95% sequence identity to a polypeptide of c).

13. A propagation material of a transgenic plant comprising the transgenic plant cell according to claim 12.

14. A transgenic plant containing heterologous DNA which encodes an EG1117 or EG307 polypeptide that is expressed in plant tissue, wherein said polypeptide is capable of increasing the yield of the plant, wherein said polypeptide is selected from the group consisting of:
a) a polypeptide encoded by a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; and SEQ ID NO:113;
b) a polypeptide encoded by a polynucleotide having at least about 95% sequence identity to a polynucleotide in a);
c) a polypeptide comprising SEQ ID NO:73; SEQ ID NO:76; SEQ ID NO:43; SEQ ID NO:46; SEQ ID NO:49; SEQ ID NO:52; SEQ ID NO:55; SEQ ID NO:58; SEQ ID NO:61; SEQ ID NO:64; SEQ ID NO:67; SEQ ID NO:70; SEQ ID NO:118; SEQ ID NO:120; SEQ ID NO:116; SEQ ID NO:96; SEQ ID NO:99; SEQ ID NO:102; SEQ ID NO:104; SEQ ID NO:106; SEQ ID NO:108; SEQ ID NO:110; SEQ ID NO:112; and SEQ ID NO:114; and
d) a polypeptide having at least about 95% sequence identity to a polypeptide of c) and which confers substantially the same yield as a polypeptide of c).

15. An isolated polynucleotide which includes a promoter operably linked to a polynucleotide that encodes an EG1117 or EG307 gene in plant tissue wherein said polynucleotide is capable of increasing the yield of a plant, wherein said polynucleotide is selected from the group consisting of:
a) a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; and SEQ ID NO:113; and
b) a polynucleotide having at least about 95% sequence identity to a polynucleotide in a).

16. The isolated polynucleotide of claim 15, wherein said polynucleotide is a recombinant polynucleotide.

17. (canceled)

18. A transfected host cell comprising a host cell transfected with a construct comprising a promoter, enhancer or intron polynucleotide from an EG1117 or EG307 polynucleotide or any combination thereof, operably linked to a polynucleotide encoding a reporter protein, wherein said EG1117 or EG307 polynucleotide is capable of increasing the yield of a plant, wherein said EG1117 or EG307 polynucleotide is selected from the group consisting of:
a) a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; and SEQ ID NO:113; and
b) a polynucleotide having at least about 95% sequence identity to a polynucleotide in a).

19. A method of determining whether a plant has a particular polynucleotide sequence comprising an EG1117 sequence, comprising the steps of:
a) comparing at least about a portion of the polynucleotide sequence of said plant with a polynucleotide sequence selected from the group consisting of (i) a polynucleotide selected from the group consisting of SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; and SEQ ID NO:93; and (ii) a polynucleotide having at least about 95% sequence identity to a polynucleotide of (i) and which confers substantially the same yield as a polynucleotide of (i), wherein one or more of the polynucleotides of a) is the particular polynucleotide; and
b) identifying whether the plant contains the particular polynucleotide.

20. The method of claim 19, wherein the plant polynucleotide sequence is genomic DNA.

21. The method of claim 19, wherein the plant polynucleotide sequence is cDNA.

22-23. (canceled)

24. The method of claim 19, wherein the plant is selected from the group consisting of Zea mays mays, Oryza sativa, Triticum aestivum, Hordeum vulgare, Saccharum officinarum, Sorghum bicolor, and Pennisetum typhoides.

25. (canceled)

26. A method of determining whether a plant has a particular polynucleotide sequence comprising an EG307 sequence, comprising the steps of:
a) comparing at least about a portion of polypeptide-coding nucleotide sequence of said plant with a polynucleotide sequence selected from the group consisting of (i) a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; and SEQ ID NO:85; and (ii) a polynucleotide having at least about 95% sequence identity to a polynucleotide of (i) and which confers substantially the same yield as a polynucleotide of (i), wherein one or more of the polynucleotides of a) is the particular polynucleotide; and
b) identifying whether the plant contains the particular polynucleotide.

27. The method of claim 26, wherein the plant polynucleotide sequence is genomic DNA.

28. The method of claim 26, wherein the plant polynucleotide sequence is cDNA.

29-30. (canceled)

31. The method of claim 26, wherein the plant is selected from the group consisting of Zea mays mays, Oryza sativa, Triticum aestivum, Hordeum vulgare, Saccharum officinarum, Sorghum bicolor, and Pennisetum typhoides.

32. (canceled)

33. A method of marker assisted breeding of plants for a particular EG1117 polynucleotide sequence, comprising the steps of:
a) comparing, for at least one plant, at least a portion of the nucleotide sequence of said plants with the particular EG1117 polynucleotide sequence selected from the group consisting of (i) a polynucleotide selected from the group consisting of SEQ ID NO:94; SEQ ID NO:95; SEQ ID NO:97; SEQ ID NO:98; SEQ ID NO:100; SEQ ID NO:101; SEQ ID NO:103; SEQ ID NO:105; SEQ ID NO:107; SEQ ID NO:109; SEQ ID NO:111; SEQ ID NO:113; SEQ ID NO:86; SEQ ID NO:87; SEQ ID NO:88; SEQ ID NO:89; SEQ ID NO:90; SEQ ID NO:91; SEQ ID NO:92; and SEQ ID NO:93; and (ii) a polynucleotide having at least about 95% sequence identity to a polynucleotide of (i) and which confers substantially the same yield as a polypeptide of (i);
b) identifying whether the plant comprises the particular polynucleotide sequence; and
c) breeding a plant comprising the particular polynucleotide sequence to produce progeny.

34. The method of claim 33, wherein the plant polynucleotide sequence is genomic DNA.

35. The method of claim 33, wherein the plant polynucleotide sequence is cDNA.

36-37. (canceled)

38. The method of claim 33, wherein the plant is selected from the group consisting of Zea mays mays, Oryza sativa, Triticum aestivum, Hordeum vulgare, Saccharum officinarum, Sorghum bicolor, and Pennisetum typhoides.

39. (canceled)

40. A method of marker assisted breeding of plants for a particular EG307 polynucleotide sequence, comprising the steps of:
a) comparing, for at least one plant, at least a portion of the nucleotide sequence of said plants with a particular EG307 polynucleotide sequence selected from the group consisting of (i) a polynucleotide selected from the group consisting of SEQ ID NO:71; SEQ ID NO:72; SEQ ID NO:74; SEQ ID NO:75; SEQ ID NO:41; SEQ ID NO:42; SEQ ID NO:44; SEQ ID NO:45; SEQ ID NO:47; SEQ ID NO:48; SEQ ID NO:50; SEQ ID NO:51; SEQ ID NO:53; SEQ ID NO:54; SEQ ID NO:56; SEQ ID NO:57; SEQ ID NO:59; SEQ ID NO:60; SEQ ID NO:62; SEQ ID NO:63; SEQ ID NO:65; SEQ ID NO:66; SEQ ID NO:68; SEQ ID NO:69; SEQ ID NO:117; SEQ ID NO:119; SEQ ID NO:115; SEQ ID NO:78; SEQ ID NO:79; SEQ ID NO:80; SEQ ID NO:81; SEQ ID NO:82; SEQ ID NO:83; SEQ ID NO:84; and SEQ ID NO:85; and (ii) a polynucleotide having at least about 95% sequence identity to a polynucleotide of (i) and which confers substantially the same yield as a polypeptide of (i);
b) identifying whether the plant comprises the particular polynucleotide sequence; and
c) breeding a plant comprising the particular polynucleotide sequence to produce progeny.

41. The method of claim 38, wherein the plant polynucleotide sequence is genomic DNA.

42. The method of claim 38, wherein the plant polynucleotide sequence is cDNA.

43-44. (canceled)

45-46. (canceled)
